(19) United States
(12) Patent Application Publication
(10) Pub. No.: US 2005/0089972 A1
(43) Pub. Date: Apr. 28, 2005

(54) Production of porphyrins

(76) Inventors: Claudia Schmidt-Dannert, Shoreview, MN (US); Arjo L. De Boer, GK Groningen (NL); Seok Joon Kwon, Pittsburgh, PA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/903,921

(22) Filed: Jul. 30, 2004

Related U.S. Application Data
(60) Provisional application No. 60/491,810, filed on Aug. 1, 2003.

(51) Int. Cl. C12P 13/04; C12P 17/12; C12N 1/21

(52) U.S. Cl. 435/106; 435/252.33; 435/252.34

(57) ABSTRACT

Methods and materials for producing porphyrins are described. In particular, microorganisms that contain one or more exogenous nucleic acids are described that produce porphyrins in high yield.

### RELATED APPLICATIONS

This application claims the benefit of U.S. Provisional Application Ser. No. 60/491,810, filed Aug. 1, 2003.

### STATEMENT AS TO FEDERALLY SPONSORED RESEARCH

Funding for the work described herein was provided in part by the federal government, grant number 1-R01-GM65471-01. The federal government may have certain rights in the invention.

### TECHNICAL FIELD

This invention relates to production of porphyrins, and more particularly to production of porphyrins in engineered microorganisms.

### BACKGROUND

Porphyrins and other related tetrapyrrolic pigments are naturally occurring molecules that play an important role in various biological processes. For example, heme (i.e., iron(II) protoporphyrin-IX complex) is the prosthetic group in hemoglobin and myoglobin, and also can be found in peroxidase and catalase. Other heme-containing proteins include the cytochromes, which serve as one-electron carriers in the electron transport chain. Porphyrins are of interest for biomedical applications, including treating cancer, psoriasis, and viral infections, gene regulation therapies, and drug targeting. Other applications of porphyrin are in material science and chemistry. Metalloporphyrins are useful in, for example, electro-catalysis, as electrodes in fuel cells, or as chemical sensors.

Porphyrins can be chemically synthesized by either total synthesis or functional derivatization of heme or chlorophyll derivatives. However, many regio- and stereoselective functionalizations of porphyrins cannot be achieved chemically. Furthermore, chemical synthesis of porphyrin derivatives usually requires several steps resulting in low overall yields, which makes porphyrins very expensive compounds.

### SUMMARY

The invention is based on a versatile system for the tailored production of porphyrins that occur as intermediates in heme biosynthesis. As described herein, different porphyrins can be produced in high yield in microorganisms such as E. coli by systematic extension of a heme biosynthetic pathway assembled from combinations of three to eight genes obtained from various microorganisms. The overproduced porphyrins may serve as “starter” structures for the production of novel, unnatural porphyrins, whereas the genes may be altered to encode enzymes with new catalytic activities. A particular gene of the assembled pathway may be modified at will, while the native chromosomal copy ensures unaffected functional heme biosynthesis for essential metabolic processes.

In one aspect, the invention features a microorganism that includes a) a genomic disruption of at least a portion of a hemC sequence, wherein the genomic disruption renders the hemC sequence non-functional; b) a genomic disruption of at least a portion of an hemD sequence, wherein the genomic disruption renders the hemD sequence non-functional; and c) an exogenous hemin uptake system, wherein the microorganism is hemin-permeable. The hemin uptake system can include an outer membrane receptor and can be inducible. The microorganism can be a Gram negative or Gram positive bacteria. The bacteria can be Escherichia coli, a species of Pseudomonas, or a species of Propionibacterium. The microorganism further can include an exogenous nucleic acid encoding a porphobilinogen deaminase polypeptide (e.g., a truncated porphobilinogen deaminase polypeptide). In some embodiments, the exogenous nucleic acid encodes the α and ω domains of the porphobilinogen deaminase polypeptide. The microorganism further can include an exogenous nucleic acid encoding an uroporphyrinogen III synthase.

In another aspect, the invention features a microorganism that includes at least one exogenous nucleic acid encoding a 5-aminolaevulinate (ALA) synthase polypeptide, an ALA dehydratase polypeptide, and a porphobilinogen deaminase polypeptide, wherein the microorganism produces an amount of uroporphyrin I that is increased relative to that of a corresponding microorganism without the at least one exogenous nucleic acid. The at least one exogenous nucleic acid further can encode an uroporphyrinogen III synthase or an uroporphyrinogen decarboxylase polypeptide, wherein the microorganism further produces an amount of uroporphyrin III or an amount of pentacarboxyporphyrin and coproporphyrin I, respectively, that is increased relative to that of a corresponding microorganism without the at least one exogenous nucleic acid.

The invention also features a microorganism that includes at least one exogenous nucleic acid that encodes a 5-ALA synthase polypeptide, an ALA dehydratase polypeptide, a porphobilinogen deaminase polypeptide, an uroporphyrinogen III synthase polypeptide, and an uroporphyrinogen decarboxylase polypeptide, wherein the microorganism produces an amount of coproporphyrin III that is increased relative to that of a corresponding microorganism without the at least one exogenous nucleic acid. The at least one exogenous nucleic acid further can encode a coproporphyrinogen III oxidase polypeptide, wherein the microorganism produces an amount of protoporphyrin IX that is increased relative to that of a corresponding microorganism without the at least one exogenous nucleic acid. The at least one exogenous nucleic acid further can encode a ferrochelatase polypeptide, wherein the microorganism produces an amount of protoporphyrin IX that is increased relative to that of a corresponding microorganism without the at least one exogenous nucleic acid. The at least one exogenous nucleic acid further can encode a metal ion transporter polypeptide, wherein the microorganism produces an amount of Zn-protoporphyrin IX that is increased relative to that of a corresponding microorganism without the at least one exogenous nucleic acid. The microorganism can be a Gram negative or Gram positive bacteria. The bacteria can be Escherichia coli, a species of Pseudomonas, or a species of Propionibacterium.

The ferrous ion chelation activity of the ferrochelatase polypeptide can be enhanced relative to a wild-type B. subtilis ferrochelatase. The ferrochelatase polypeptide can include one or more mutations at amino acid residues selected from the group consisting of residues 11, 31, 61, 76, 102, 104, 184, 185, 212, and 302 of SEQ ID NO:7 (e.g., a mutation at residues 76 and 102 of SEQ ID NO:7, a mutation at residues 11 and 104 of SEQ ID NO:7, or a mutation at residues 61, 185, and 212 of SEQ ID NO:7).

In another aspect, the invention features a microorganism that includes an exogenous nucleic acid encoding a 5-ALA synthase polypeptide, an ALA dehydratase polypeptide, a porphobilinogen deaminase polypeptide, an uroporphyrinogen III synthase polypeptide, and a protoporphyrinogen oxidase polypeptide or a coproporphyrinogen III oxidase polypeptide, wherein the microorganism produces an amount of uroporphyrin III that is increased relative to that of a corresponding microorganism without the at least one exogenous nucleic acid.

In another aspect, the invention features a microorganism that includes an exogenous nucleic acid encoding a 5-ALA synthase polypeptide, an ALA dehydratase polypeptide, a porphobilinogen deaminase polypeptide, an uroporphyrinogen decarboxylase polypeptide, and a protoporphyrinogen oxidase polypeptide, wherein the microorganism further produces an amount of pentacarboxyporphyrin that is increased relative to that of a corresponding microorganism without the at least one exogenous nucleic acid.

In yet another aspect, the invention features a microorganism that includes an exogenous nucleic acid encoding a 5-ALA synthase polypeptide, an ALA dehydratase polypeptide, a porphobilinogen deaminase polypeptide, an uroporphyrinogen III synthase polypeptide, an uroporphyrinogen decarboxylase polypeptide, and a protoporphyrinogen oxidase polypeptide, wherein the microorganism produces an amount of coproporphyrin III that is increased relative to that of a corresponding microorganism without the at least one exogenous nucleic acid. The at least one exogenous nucleic acid further can encode a a protoporphyrinogen oxidase polypeptide, wherein the microorganism produces an amount of coproporphyrin III and protoporphyrin IX that is increased relative to that of a corresponding microorganism without the at least one exogenous nucleic acid.

The invention also features an isolated nucleic acid encoding a ferrochelatase polypeptide, wherein the polypeptide comprises one or more mutations at amino acid residues selected from the group consisting of residues 11, 31, 61, 76, 102, 104, 184, 185, 212, and 302 of SEQ ID NO:7. The chelation activity of the polypeptide can be enhanced relative to a ferrochelatase polypeptide having the amino acid sequence of SEQ ID NO:7. The ferrochelatase polypeptide can include a mutation at residues 76 and 102 of SEQ ID NO:7, a mutation at residues 11 and 104 of SEQ ID NO:7, or a mutation at residues 61, 185, and 212 of SEQ ID NO:7. The invention also features a vector that includes such isolated nucleic acids and host cells that include the vector.

In yet another aspect, the invention features a method of identifying variant ferrochelatase polypeptides. The method includes a) providing a population of microorganisms expressing a variant ferrochelatase polypeptide, wherein a plurality of microorganisms within the population differ from one another in that each microorganism in the plurality expresses a different ferrochelatase polypeptide, wherein microorganisms within the population are capable of producing protoporphyrin IX; b) culturing the population of microorganisms in the presence of at least one metal ion; and c) identifying microorganisms from the population that have the ability to incorporate the at least one metal ion into protoporphyrin IX. Microorganisms in the population can express an ALA synthase polypeptide, an ALA dehydratase polypeptide, a porphobilinogen deaminase polypeptide, an uroporphyrinogen synthase polypeptide, an uroporphyrinogen decarboxylase polypeptide, a coproporphyrinogen oxidase polypeptide, and a metal ion transporter polypeptide. The identifying step can include fluorescence activated cell sorting.

The invention also features a microorganism that includes at least one exogenous nucleic acid encoding a 5-ALA synthase polypeptide, an ALA dehydratase polypeptide, a porphobilinogen deaminase polypeptide, an uroporphyrinogen III synthase polypeptide, an uroporphyrinogen decarboxylase polypeptide, a coproporphyrinogen III oxidase polypeptide, a ferrochelatase polypeptide, and a metal ion transporter polypeptide, wherein the microorganism produces an amount of Zn-protoporphyrin IX that is increased relative to that of a corresponding microorganism without the at least one exogenous nucleic acid. The microorganism can be a Gram negative or Gram positive bacteria. The bacteria can be Escherichia coli, a species of Pseudomonas, or a species of Propionibacterium. The ferrous ion chelation activity of the ferrochelatase polypeptide can be enhanced relative to a wild-type B. subtilis ferrochelatase polypeptide. The ferrochelatase polypeptide can include one or more mutations at amino acid residues selected from the group consisting of residues 11, 31, 61, 76, 102, 104, 184, 185, 212, and 302 of SEQ ID NO:7 (e.g., a mutation at residues 76 and 102 of SEQ ID NO:7, a mutation at residues 11 and 104 of SEQ ID NO:7, or a mutation at residues 61, 185, and 212 of SEQ ID NO:7). The metal ion transporter can be a zinc and iron transporter such as a ZupT polypeptide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic of the assembled pathway of porphyrin biosynthesis in engineered E. coli. Abbreviation: Gly, glycine; ALA, 5-aminolevulinic acid; PBG, porphobilinogen; HMB, 1-hydroxymethylbilane; Uro III, uroporphyrin III; Copro III, coproporphyrin III; Proto IX, protoporphyrin IX; Uro I, uroporphyrin I, PCP, pentacarboxyporphyrin, Copro I, coproporphyrin I. All porphyrins were derived from the corresponding porphyrinogens. *OX means spontaneous oxidation during and after extraction of E. coli transformants.

FIG. 2 is a chromatogram of porphyrin extracts from E. coli cells overexpressing hemA, hemB and hemC (combination 5 in Table 2, (light line) and hemA, hemB, hemC and hemD (combination 6 in Table 2 (dark line)). The insets show magnifications of elution profile of the uroporphyrin isomers I and III, and coproporphyrin isomers I and III, respectively.

FIG. 3 is a graph depicting the results of an assay for zinc-chelatase activity of B. subtilis ferrochelatase hemH expressed in E. coli cells. Zinc chelatase was assayed using a cell-free extract of E. coli cells overexpressing hemA through hemH (combination 10 in Table 2). Extracts without overexpressed hemH (combination 9 in Table 2 (◯) and empty vector combination 1 in Table 2 (●)) were used as controls. A control without cell-free extract was also included (∇).

FIG. 4 is a schematic representation of the pSPLIT series of plasmids. The hemC gene was split into two parts by insertion of two stop codons (denoted by asterisks) followed by an RBS (italic) and a start codon (bold). A SalI site (underlined) was introduced simultaneously. The pTRUNC series was derived from the pSPLIT-series by deleting the SalI-NotI fragment.

FIG. 5 is a ribbon structure of hemC, with the positions of the dissections and truncations marked.

FIG. 6A and 6B are schematics of plasmids pUCmod-zupT (A) and pBBR-hemEFzupT (B).

FIG. 7A and 7B are HPLC chromatograms of E. coli transformants containing hemABCDEF genes (A) and hemABCDEFH plus zupT genes (B).

FIG. 8A and 8B depict Zn-protop IX characteristics in HPLC mobile phase. FIG. 8A depicts the Q band spectrum. FIG. 8B depicts the electrospray ionization (ESI)-Mass spectrum.

FIG. 9A and 9B are a dot plot and histogram, respectively, of flow cytometric discrimination of E. coli JM109 cells on the basis of porphyrin production. Population 1. Control E. coli cells; Population 2. Zn-protop IX producing E. coli (hemABCDEFH+zupT); Population 3. Protoporphyrin IX producing E. coli (hemABCDEF).

FIG. 10 is a FACS histogram of the proto IX producing E. coli cells pUC-hemH library (Population 1), pUC-hemH168 (Population 2), and pUC-hemH125 (Population 3).

FIG. 11A, 11B, and 11C are HPLC profiles of the proto IX producing E. coli cells containing wild type ferrochelatases from B. halodurans C-125 (A), B. subtilis 168 (B), and mutant 6 (C). 1. heme; 2. Proto IX.

FIG. 12 contains the nucleotide sequence of the hemH gene from B. subtilis 168 and the amino acid sequence of the encoded ferrochelatase polypeptide.

### DETAILED DESCRIPTION

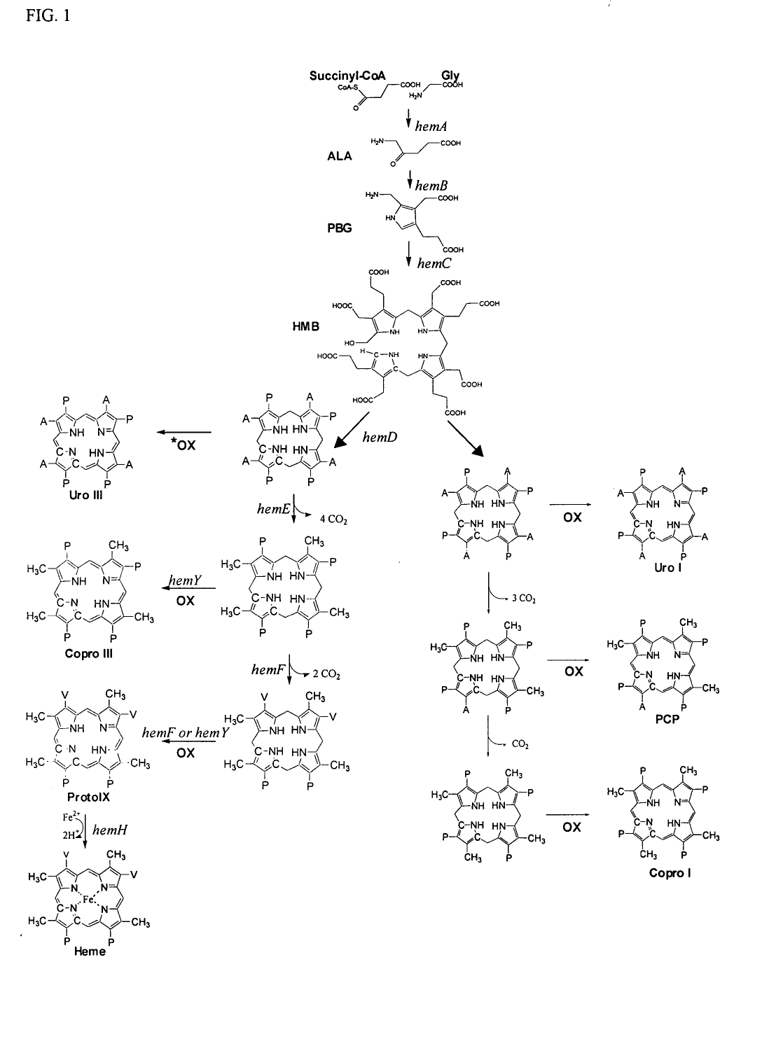

In general, the invention provides methods and materials for producing porphyrins in microorganisms. As used herein, the term “porphyrin” refers to both the oxidized and reduced (i.e., porphyrinogen) forms of the porphyrin. The term “metalloporphyrin” indicates that a metal ion has been complexed with the porphyrin. As shown in FIG. 1, tetrapyrrole biosynthesis starts with the synthesis of ALA. Two molecules of aminolevulinic acid (ALA) are then condensed to form porphobilinogen (PBG). In turn, four molecules of PBG are condensed to 1-hydroxymethylbilane (HMB), which is subsequently cyclized to uroporphyrinogen III (uro'gen III) whereby ring D undergoes inversion. Direct coupling of these two enzymatic steps prevents spontaneous formation of the uro'gen I isomer, which is not a substrate of most subsequent biosynthetic enzymes. The next step in heme biosynthesis involves the oxidative decarboxylation of uro'gen III to form coproporphyrinogen III (copro'gen III), followed by oxidation reactions to synthesize protoporphyrin IX. Finally, insertion of a ferrous iron ion into protoporphyrin IX produces protoheme IX.

Isolated Nucleic Acids

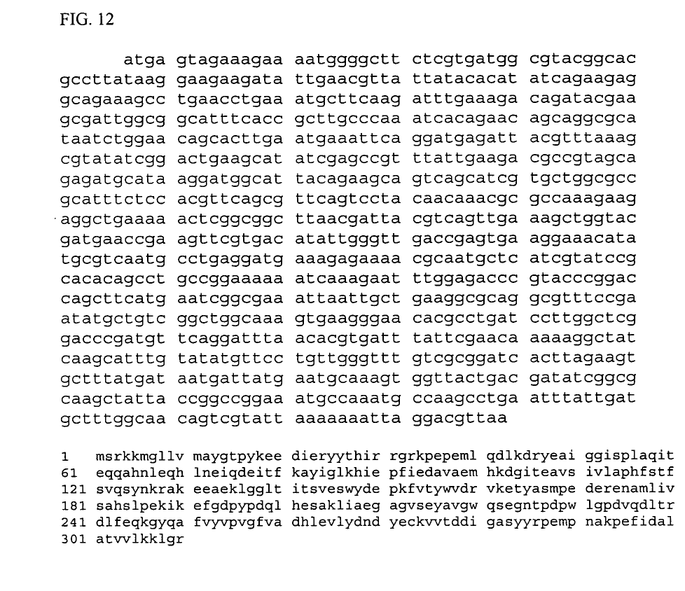

The invention features isolated nucleic acids that encode variant ferrochelatase polypeptides. As used herein, variant ferrochelatase polypeptides have one or more amino acid substitutions, insertions, or deletions relative to a wild-type ferrochelatase polypeptide. For example, an isolated nucleic acid can encode a ferrochelatase polypeptide from Bacillus subtilis 168 (GenBank Accession No. Z99109 (nt. 75517-76449)) having one or more amino acid substitutions, deletions, or insertions. The nucleotide sequence of the B. subtilis 168 hemH gene is set forth in SEQ ID NO:6 (FIG. 12); the amino acid sequence of the B. subtilis 168 ferrochelatase is set forth in SEQ ID NO:7 (FIG. 12). As used herein, the term “polypeptide” refers to any chain of amino acid residues, regardless of length or post-translational modification, that has the desired catalytic activity. For example, a ferrochelatase polypeptide refers to any chain of amino acid residues, regardless of length or post-translation modification, that has the ability to incorporate a metal ion into protoporphyrin IX.

As used herein, the term “nucleic acid” refers to both RNA and DNA, including cDNA, genomic DNA, synthetic (e.g., chemically synthesized) DNA, and DNA containing nucleic acid analogs. Nucleotides are referred to herein by the standard one-letter designation (A, C, G, or T). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine or 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See Summerton and Weller, Antisense Nucleic Acid Drug Dev. (1997) 7(3):187-195; and Hyrup et al. (1996) Bioorgan. Med. Chem. 4(1):5-23. In addition, the deoxyphosphate backbone can be replaced with, for example, a phosphorothioate or phosphorodithioate backbone, a phosphoroamidite, or an alkyl phosphotriester backbone. The nucleic acid can be double-stranded or single-stranded (i.e., a sense or an antisense single strand).

As used herein, “isolated nucleic acid” refers to a nucleic acid that is separated from other nucleic acid molecules that are present in a genome, including nucleic acids that normally flank one or both sides of the nucleic acid in the genome (e.g., nucleic acids that flank the hemH gene). The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

As described herein, isolated hemH nucleic acid molecules are at least 10 nucleotides in length. For example, the nucleic acid can be about 10, 10-20 (e.g., 10, 11, 12, 13, 14, 15, 16, 17, 18, 19, or 20 nucleotides in length), 20-50, 50-100 or greater than 100 nucleotides in length (e.g., greater than 150, 200, 250, 300, 400, 500, 600, 700, 800, 900, or more nucleotides in length). The full-length B. subtilis 168 hemH transcript is 933 nucleotides in length. Nucleotide sequence variants can be, for example, deletions, insertions, or substitutions at one or more nucleotide positions (e.g., 1, 2, 3, 4, 5, 6, 7, 8, 9, 10, 15, 20, 30, or more than 30 positions), provided that the nucleic acid is at least 60% identical (e.g., 65%, 70%, 75%, 80%, 85%, 90%, 95%, 98%, or 99% identical) over its length to the corresponding region of the wild-type sequence (e.g., the wild-type sequence set forth in SEQ ID NO:6, FIG. 12). Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. To determine percent sequence identity, a target nucleic acid sequence is compared to the identified nucleic acid sequence using the BLAST 2 Sequences (Bl2seq) program from the stand-alone version of BLASTZ containing BLASTN version 2.0.14. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the Bl2seq program can be found in the readme file accompanying BLASTZ.

Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. The following command will generate an output file containing a comparison between two sequences: C:\Bl2seq -i c:\seq1.txt -j c:\seq2.txt -p blastn -o c:\output.txt -q −1 -r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length followed by multiplying the resulting value by 100. For example, if (1) a 1000 nucleotide target sequence is compared to the sequence set forth in SEQ ID NO:1, (2) the Bl2seq program presents 200 nucleotides from the target sequence aligned with a region of the sequence set forth in SEQ ID NO:1 where the first and last nucleotides of that 200 nucleotide region are matches, and (3) the number of matches over those 200 aligned nucleotides is 180, then the 1000 nucleotide target sequence contains a length of 200 and a percent identity over that length of 90 (i.e., 180 ) 200×100=90).

It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

Isolated nucleic acid molecules of the invention can be produced by standard techniques, including, without limitation, common molecular cloning and chemical nucleic acid synthesis techniques. For example, polymerase chain reaction (PCR) techniques can be used to obtain an isolated nucleic acid containing a hemH nucleotide sequence variant. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication, or nucleic acid sequence-based amplification also can be used to obtain isolated nucleic acids. See, for example, Lewis Genetic Engineering News, 12(9):1 (1992); Guatelli et al., Proc. Natl. Acad. Sci. USA, 87:1874-1878 (1990); and Weiss, Science, 254:1292 (1991).

Isolated nucleic acids of the invention also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector if desired.

Isolated nucleic acids of the invention also can be obtained by mutagenesis. For example, the sequences set forth in SEQ ID NO:6 can be mutated using standard techniques including oligonucleotide-directed mutagenesis and site-directed mutagenesis through PCR. See Short Protocols in Molecular Biology, Chapter 8, Green Publishing Associates and John Wiley & Sons, edited by Ausubel et al., 1992. Other techniques for obtaining isolated nucleic acids of the invention include error prone PCR, DNA shuffling, random mutagenesis, or recombination and selection. See for example, Stemmer, Nature (1994) 370:389-391 and Crameri et al., Nature (1998) 391:288-291.

Ferrochelatase Polypeptides

The invention provides purified variant ferrochelatase polypeptides that are encoded by the nucleic acid molecules of the invention. Variant ferrochelatase polypeptides have amino acid sequences that differ from the amino acid sequences of the corresponding wild-type ferrochelatase polypeptides. As used herein, an amino acid sequence variant refers to a deletion, insertion, or substitution at one or more amino acid positions (e.g., 1, 2, 3, 10, or more-than 10 positions). For example, a purified variant ferrochelatase polypeptide can have an amino acid substitution at one or more of amino acid residues 11, 31, 61, 76, 102, 104, 184, 185, 212, or 302 of the amino acid sequence of the B. subtilis 168 ferrochelatase (SEQ ID NO:7; FIG. 12). In particular, a valine can be substituted for methionine at residue 11, a glycine can be substituted for an arginine at residue 31, a lysine can be substituted for a glutamic acid at residue 61, a glycine can be substituted for an aspartic acid at residue 76, a threonine can be substituted for a lysine at residue 102, an alanine can be substituted for a glycine at residue 104, a glutamine can be substituted for a leucine at residue 185, an aspartic acid can be substituted for a glycine at residue 212, and an alanine can be substituted for a threonine at residue 302. In some embodiments, the B. subtilis 168 ferrochelatase polypeptide (SEQ ID NO:7) can have a mutation at residues 76 and 102 (e.g., D76G and K102T); a mutation at residues 11 and 104 (e.g., M11V and G104A); or a mutation at residues 61, 184 or 185, or 212 (e.g., E61K, L185Q, G212D). A ferrochelatase polypeptide variant may have one or more additional sequence variants in addition to the variants described previously, provided that the polypeptide has an amino acid sequence that is at least 60% identical (e.g., 65%, 70%, 75%, 80%, 85%, 90%, 95%, or 99% identical) over its length to the sequence set forth in SEQ ID NO:7.

Percent sequence identity is calculated by determining the number of matched positions in aligned amino acid sequences, dividing the number of matched positions by the total number of aligned amino acids, and multiplying by 100. The percent identity between amino acid sequences therefore is calculated in a manner analogous to the method for calculating the identity between nucleic acid sequences, using the Bl2seq program from the stand-alone version of BLASTZ containing BLASTN version 2.0.14 and BLASTP version 2.0.14; see above section on nucleic acids. A matched position refers to a position in which identical residues occur at the same position in aligned amino acid sequences. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. The following command will generate an output file containing a comparison between two amino acid sequences: C:\Bl2seq -i c:\seq1.txt -j c:\seq2.txt -p blastp -o c:\output.txt. If the target sequence shares homology with any portion of the identified sequence, then the designated output file will present-those regions of homology as aligned sequences. If the target sequence does not share homology with any portion of the identified sequence, then the designated output file will not present aligned sequences. Once aligned, a length and a percent identity over that length can be determined as described above.

Amino acid substitutions may be conservative or non-conservative. Conservative amino acid substitutions replace an amino acid with an amino acid of the same class, whereas non-conservative amino acid substitutions replace an amino acid with an amino acid of a different class. Conservative amino acid substitutions typically have little effect on the structure or function of a polypeptide. Examples of conservative substitutions include amino acid substitutions within the following groups: glycine and alanine; valine, isoleucine, and leucine; aspartic acid and glutamic acid; asparagine, glutamine, serine, and threonine; lysine, histidine, and arginine; and phenylalanine and tyrosine.

Non-conservative substitutions may result in a substantial change in the hydrophobicity of the polypeptide or in the bulk of a residue side chain. In addition, non-conservative substitutions may make a substantial change in the charge of the polypeptide, such as reducing electropositive charges or introducing electronegative charges. Examples of non-conservative substitutions include a basic amino acid for a non-polar amino acid, or a polar amino acid for an acidic amino acid.

In some embodiments, a substitution can be within the substrate-binding pocket of the ferrochelatase polypeptide. For example, within the B. subtilis 168 ferrochelatase polypeptide, residues 184 or 185 can be altered. These residues are located above pyrrole rings B and C, and are thought to bend pyrrole rings B and C.

The term “purified” as used herein with reference to a polypeptide refers to a polypeptide that either has no naturally occurring counterpart (e.g., a peptidomimetic), has been chemically synthesized and is thus uncontaminated by other polypeptides, or has been separated or purified from other cellular components by which it is naturally accompanied (e.g., other cellular proteins, polynucleotides, or cellular components). Typically, the polypeptide is considered “purified” when it is at least 70% (e.g., 70%, 80%, 90%, 95%, or 99%), by dry weight, free from the proteins and naturally occurring organic molecules with which it naturally associates.

In some embodiments, an activity of a variant ferrochelatase polypeptide can be altered relative to the corresponding wild-type ferrochelatase polypeptide. Ferrochelatase predominantly inserts Fe²⁺ into protoporphyrin IX, although it also can insert other metal ions such as Co²⁺, Ni²⁺, and Zn²⁺ at a lower rate. The activity of the variant ferrochelatase polypeptide can be reduced or enhanced (e.g., enhanced rate of incorporation of Fe²⁺, Co²⁺, Ni²⁺, or Zn²⁺ into protoporphyrin IX), or the activity may be a different activity (e.g., incorporation of a metal ion typically not incorporated by ferrochelatase such as Cu²⁺ or Mn²⁺).

Activity of variant ferrochelatase polypeptides can be assessed in vitro (e.g., an in vitro enzyme assay, such as that described by Camadro and Labbe (1988) J. Biol. Chem. 263:11675-82) or in vivo. Porphyrins and metalloporphyrins produced by a microorganism expressing the entire heme pathway can be analyzed by HPLC. Alternatively, as described herein, a high-throughput screening method based on FACS (fluorescent activated cell sorting) of microorganisms overproducing porphyrins and metalloporphyrins can be used. In this method, a population of microorganisms that are expressing variant ferrochelatase polypeptides and are capable of producing protoporphyrin IX are used. Within the population, a plurality of microorganisms differs from one another in that each microorganism in the plurality expresses a different variant ferrochelatase polypeptide. In some embodiments, microorganisms within the population express an ALA synthase polypeptide, an ALA dehydratase polypeptide, a porphobilinogen deaminase polypeptide, an uroporphyrinogen synthase polypeptide, an uroporphyrinogen decarboxylase polypeptide, a coproporphyrinogen III oxidase polypeptide, and a metal ion transporter polypeptide. Populations of such microorganisms can be produced by transforming the microorganisms (e.g., E. coli) with one or more exogenous nucleic acids encoding the polypeptides, as discussed herein.

The population of microorganisms can be cultured under suitable conditions for protoporphyrin IX production. After the microorganisms begin to accumulate protoporphyrin IX, the appropriate metal ion(s) can be added (e.g., ZnSO₄ or FeSO₄) to the media, and the microorganisms cultivated for a period of time sufficient to incorporate the metal ion into protoporphyrin IX. Microorganisms within the population can be separated based on the distinct fluorescent properties of the porphyrins that can be produced within the microorganisms. As such, the method provides a high-throughput method for identifying variant ferrochelatase polypeptides having the desired activity from a library of variant polypeptides.

Variant ferrochelatase polypeptides can be produced by a number of methods, many of which are well known-in the art. By way of example and not limitation, variant ferrochelatase polypeptides can be produced recombinantly or through chemical synthesis. For example, standard recombinant technology, using expression vectors encoding variant ferrochelatase polypeptides can be used. The resulting variant ferrochelatase polypeptides then can be purified. Expression systems that can be used for small or large scale production of variant ferrochelatase polypeptides include, without limitation, microorganisms such as bacteria (including both Gram negative and Gram positive bacteria) transformed with recombinant bacteriophage DNA, plasmid DNA, or cosmid DNA expression vectors containing the nucleic acid molecules of the invention; yeast (e.g., S. cerevisiae) transformed with recombinant yeast expression vectors containing the nucleic acid molecules of the invention; insect cell systems infected with recombinant virus expression vectors (e.g., baculovirus) containing the nucleic acid molecules of the invention; plant cell systems infected with recombinant virus expression vectors (e.g., tobacco mosaic virus) or transformed with recombinant plasmid expression vectors (e.g., Ti plasmid) containing the nucleic acid molecules of the invention; or mammalian cell systems (e.g., primary cells or immortalized cell lines such as COS cells or Chinese hamster ovary cells) harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids of the invention.

In bacterial systems, a strain of E. coli such as JM109 can be used. Suitable E. coli vectors include, but are not limited to, pUC18, pUC19, the pGEX series of vectors that produce fusion proteins with glutathione S-transferase (GST), and pBluescript series of vectors. Transformed E. coli are typically grown exponentially then stimulated with isopropylthiogalactopyranoside (IPTG) prior to harvesting. In general, fusion proteins produced from the pGEX series of vectors are soluble and can be purified easily from lysed cells by adsorption to glutathione-agarose beads followed by elution in the presence of free glutathione. The pGEX vectors are designed to include thrombin or factor Xa protease cleavage sites such that the cloned target gene product can be released from the GST moiety.

Suitable methods for purifying the polypeptides of the invention can include, for example, affinity chromatography, immunoprecipitation, size exclusion chromatography, or ion exchange chromatography. The extent of purification can be measured by any appropriate method, including but not limited to: column chromatography, polyacrylamide gel electrophoresis, or high-performance liquid chromatography. Variant ferrochelatase polypeptides also can be “engineered” to contain a tag sequence described herein that allows the polypeptide to be purified (e.g., captured onto an affinity matrix). Finally, immunoaffinity chromatography also can be used to purify variant ferrochelatase polypeptides.

Vectors and Host Cells

The invention also provides vectors containing nucleic acids such as those described above. As used herein, a “vector” is a replicon, such as a plasmid, phage, or cosmid, into which another DNA segment may be inserted so as to bring about the replication of the inserted segment. The vectors of the invention can be expression vectors. An “expression vector” is a vector that includes one or more expression control sequences, and an “expression control sequence” is a DNA sequence that controls and regulates the transcription and/or translation of another DNA sequence.

In the expression vectors of the invention, the nucleic acid is operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalovirus, retroviruses, poxviruses, adenoviruses, and adeno-associated viruses. Numerous vectors and expression systems are commercially available from such corporations as Novagen (Madison, Wisc.), Clontech (Palo Alto, Calif.), Stratagene (La Jolla, Calif.), and Invitrogen/Life Technologies (Carlsbad, Calif.).

An expression vector can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide including at either the carboxyl or amino terminus.

The invention also provides host cells containing vectors of the invention. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Suitable methods for transforming and transfecting host cells are found in Sambrook et al., Molecular Cloning: A Laboratory Manual (2^{nd} edition), Cold Spring Harbor Laboratory, NY (1989), and reagents for transformation and/or transfection are commercially available (e.g., Lipofectin (Invitrogen/Life Technologies); Fugene (Roche, Indianapolis, Ind.); and SuperFect (Qiagen, Valencia, Calif.)).

Engineered Microorganisms

Microorganisms that are suitable for producing porphyrins may or may not naturally produce porphyrins, and include both Gram-positive and Gram-negative bacteria, yeast, and filamentous fungi. For example, a suitable microorganism can be a species from one of the following genera: Aerobacter, Agrobacterium, Alcaligenes, Azotobacter, Bacillus, Clostridium, Corynebacterium, Escherichia, Flavobacterium, Micromonospora, Mycobacterium, Norcardia, Propionibacterium, Protaminobacter, Proteus, Pseudomonas, Rhizobium, Salmonella, Serratia, Streptomyces, Streptococcus, and Xanthomonas. For example, Arthrobacter hyalinus, E. coli, Propionibacterium freudenreichii or P. shermanii, Rhodopseudomonas protamicus, Pseudomonas denitrificans, Nocardia rugosa or N. gardneri, Rhizobium cobalaminogenum, Streptomyces olivaceus, or Butyribacterium methylotrophicum can be used. See, for example, Martens et al., Appl. Microbiol. Biotechnol. (2002) 58:275-285.

An engineered microorganism can include at least one exogenous nucleic acid encoding a 5-aminolaevulinate (ALA) synthase polypeptide, an ALA dehydratase polypeptide, and a porphobilinogen deaminase polypeptide. Such a microorganism produces an amount of uroporphyrin I that is increased relative to that of a corresponding microorganism without the exogenous nucleic acid. Standard analytical procedures can be used to detect the porphyrins, including adsorption of the porphyrins using DEAE-Sephadex then extraction with an organic solvent and HPLC analysis.

ALA synthase (EC 2.3.1.37) is encoded by hemA and catalyzes the condensation of succinyl CoA and glycine. A suitable nucleic acid encoding ALA synthase includes the hemA gene from Rhodobacter capsulatus (GenBank Accession No. X53309). ALA dehydratase (EC 4.2.1.24) is encoded by the hemB gene and catalyzes the dimerization of two ALA molecules to form porphobilinogen. A suitable nucleic acid encoding ALA dehydratase includes the hemB gene from E. coli (GenBank Accession No. D85613). Porphobilinogen deaminase (EC 4.3.1.8) is encoded by the hemC gene and catalyzes the deamination and polymerization of four porphobilinogens to form the unstable hydroxymethylbilane. Suitable nucleic acids encoding porphobilinogen deaminase include the hemC genes from E. coli, Synechocystis sp., and R. capsulatus (GenBank Accession Nos. 41665, 1652725, and U16796, respectively).

Exogenous nucleic acids can be integrated into the genome of the microorganism or maintained in an episomal state. In other words, microorganisms can be stably or transiently transfected with an exogenous nucleic acid. Suitable nucleic acid constructs are described above. Typically, each gene can be placed under control of a constitutive promoter to circumvent the regulatory circuitry at the transcriptional and translational level that governs porphyrin (and heme) biosynthesis in native systems.

Any method can be used to introduce an isolated nucleic acid into a microorganism. In fact, many methods for introducing nucleic acid into cells, whether in vivo or in vitro, are well known to those skilled in the art. For example, calcium phosphate precipitation, conjugation, electroporation, heat shock, lipofection, microinjection, and viral-mediated nucleic acid transfer are common methods that can be used to introduce nucleic acid molecules into cells. In addition, naked DNA can be delivered directly to cells in vivo as describe elsewhere (U.S. Pat. Nos. 5,580,859 and 5,589,466).

Any method can be used to identify microorganisms that contain an isolated nucleic acid within the scope of the invention. For example, PCR and nucleic acid hybridization techniques such as Northern and Southern analysis can be used. In some cases, immunohistochemistry and biochemical techniques can be used to determine if a microorganism contains a particular nucleic acid by detecting the expression of a polypeptide encoded by that particular nucleic acid. Enzymatic activities of the polypeptide of interest also can be detected or an end product (e.g., uroporphyrin I) can be detected as an indication that the microorganism contains the introduced nucleic acid and expresses the encoded polypeptide from that introduced nucleic acid.

The microorganisms described herein can contain a single copy or multiple copies (e.g., about 5, 10, 20, 35, 50, 75, 100 or 150 copies), of a particular exogenous nucleic acid. All non-naturally-occurring nucleic acids are considered an exogenous nucleic acid once introduced into the cell. The term “exogenous” as used herein with reference to a nucleic acid and a particular cell refers to any nucleic acid that does not originate from that particular cell as found in nature. Nucleic acid that is naturally occurring also can be exogenous to a particular cell. For example, an entire operon that is isolated from a bacterium is an exogenous nucleic acid with respect to a second bacterium once that operon is introduced into the second bacterium. In addition, the cells described herein can contain more than one particular exogenous nucleic acid (e.g., two or three exogenous nucleic acids). For example, a bacterial cell can contain about 50 copies of exogenous nucleic acid X as well as about 75 copies of exogenous nucleic acid Y and 50 copies of exogenous nucleic acid Z. In these cases, each different nucleic acid can encode a different polypeptide having its own unique enzymatic activity or encode multiple polypeptides, with each polypeptide having a distinct enzymatic activity. For example, a bacterial cell can contain two or three different exogenous nucleic acids such that a high level of a porphyrin is produced. In some embodiments, the exogenous nucleic acids are contained in plasmids belonging to different incompatibility groups to enable modular pathway assembly. In addition, a single exogenous nucleic acid can encode one or more polypeptides.

Depending on the microorganism and the metabolites present within the microorganism, one or more of the following additional polypeptides may be expressed in the microorganism in addition to ALA synthase, ALA dehydratase, and porphobilinogen deaminase: uroporphyrinogen III synthase, uroporphyrinogen decarboxylase, coproporphyrinogen III oxidase, ferrochelatase, protoporphyrinogen IX oxidase, or a metal ion transporter. Uroporphyrinogen III synthase (EC 4.2.1.75) is encoded by the hemD gene and catalyzes ring D inversion and cyclization of hydroxymethylbilane to uroporphyrinogen III (uro'gen III). A suitable nucleic acid encoding uroporphyrinogen III synthase includes the hemD gene from E. coli (GenBank Accession No. Y00883). Uroporphyrinogen decarboxylase (EC 4.1.1.37) is encoded by the hemE gene and catalyzes the decarboxylation of four acetate side-chains of uro'gen II to form coproporphyrinogen III (copro'gen III). Suitable nucleic acids encoding uroporphyrinogen decarboxylase include the hemE genes from E. coli, Synechocystis sp. (strain PCC6803), and R. capsulatus (GenBank Accession Nos. D12624, 1001291, U16796, respectively). The hemE genes from Klebsiella pneumoniae and Salmonella typhimurim also can be used. Coproporphyrinogen III oxidase (EC 1.3.3.3) is encoded by the hemF gene and catalyzes the oxidative decarboxylation of two propionate side chains in rings A and B of copro'gen III to vinyl groups to form protoporphyrinogen IX (proto'gen IX). Suitable nucleic acids encoding coproporphyrinogen III oxidase include the hemF genes from E. coli, Synchocystis sp. (strain PCC6803), Klebsiella pneumoniae and Salmonella typhimurim. See, for example, GenBank Accession Nos. 1651897. Protoporphyrinogen IX oxidase (EC 1.3.3.4) is encoded by the hemY gene and catalyzes the six-electron oxidation of proto'gen IX to form protoporphyrin IX. Suitable nucleic acids encoding protoporphyrinogen IX oxidase include the hemY genes from B. subtilis and B. halodurans (GenBank Accession Nos. M97208 and 10173727, respectively). The hemY genes from Klebsiella pneumoniae and Salmonella typhimurim also can be used. Ferrochelatase (EC 4.99.1.1) is encoded by hemH and catalyzes Fe²⁺ metallation of protoporphyrin IX to produce protohaem IX. Suitable nucleic acids encoding ferrochelatase include the hemH genes from E. coli, R. capsulatus, and B. subtilis (GenBank Accession Nos. AE000153, U34391, and M97208, respectively). A suitable metal ion transporter polypeptide includes ZupT, a member of the ZIP (zinc and iron transporter) metal transporter family. See, Guerinot, (2000) Biophys.Acta 1465:190-198. ZupT is a broad-range metal ion transporter (Grass et al, 2002 J. Bacteriol. 184:864-66) and is encoded by the ygiE gene (zupT). See, GenBank Accession No. AE000386 for the ygiE gene from E. coli.

For example, a microorganism can include at least one exogenous nucleic acid that encodes an ALA synthase polypeptide, ALA dehydratase polypeptide, porphobilinogen deaminase polypeptide and an uroporphyrinogen III synthase polypeptide and/or an uroporphyrinogen decarboxylase polypeptide. Such a microorganism can produce increased amounts of uroporphyrin III, pentacarboxyporphyrin and coproporphyrin I, or coproporphyrin III relative to that of a corresponding microorganism without the exogenous nucleic acid.

In other embodiments, a microorganism can include at least one exogenous nucleic acid that encodes an ALA synthase polypeptide, ALA dehydratase polypeptide, porphobilinogen deaminase polypeptide, an uroporphyrinogen III synthase polypeptide, an uroporphyrinogen decarboxylase polypeptide, and a coproporphyrinogen III oxidase polypeptide. Such a microorganism can produce an increased amount of protoporphyrin IX that is increased relative to that of a corresponding microorganism without the exogenous nucleic acid. In embodiments in which the exogenous nucleic acid further encodes a ferrochelatase polypeptide, the microorganism can produce an amount of protoporphyrin IX that is increased relative to that of a corresponding microorganism without the exogenous nucleic acid. The ferrochelatase polypeptide can have increased ferrous ion chelation activity, as discussed above.

Increased amounts of uroporphyrin III can be produced in a microorganism by introducing an exogenous nucleic acid that encodes an ALA synthase polypeptide, ALA dehydratase polypeptide, porphobilinogen deaminase polypeptide, an uroporphyrinogen III synthase polypeptide, and a protoporphyrinogen oxidase polypeptide or a coproporphyrinogen III oxidase polypeptide. Uroporphyrin III is the common precursor of all tetrapyrroles in living cells at which a major branching of the biosynthetic pathways occurs.

Increased amounts of pentacarboxyporphyrin can be produced in a microorganism by using an exogenous nucleic acid encoding an ALA synthase polypeptide, an ALA dehydratase polypeptide, a porphobilinogen deaminase polypeptide, an uroporphyrinogen decarboxylase polypeptide, and a protoporphyrinogen oxidase polypeptide.

Similarly, increased amounts of coproporphyrin III can be produced in a microorganism by introducing at least one exogenous nucleic acid encoding an ALA synthase polypeptide, an ALA dehydratase polypeptide, a porphobilinogen deaminase polypeptide, an uroporphyrinogen III synthase polypeptide, an uroporphyrinogen decarboxylase polypeptide, and a protoporphyrinogen oxidase polypeptide. In embodiments in which the exogenous nucleic acid further encodes a coproporphyrinogen III oxidase, increased amounts of coproporphyrin III and protoporphyrin IX. Further expression of a ferrochelatase and a metal ion transporter such as ZupT allows increased production of Zn-protoporphyrin IX.

In other embodiments, a microorganism has reduced enzymatic activity (e.g., reduced porphobilinogen deaminase and/or uroporphyrinogen III synthase activity). The term “reduced” as used herein with respect to a microorganism and a particular activity (e.g., particular enzymatic activity) refers to a lower level of activity than that measured in a corresponding microorganism. Such reduced enzymatic activities can be the result of lower enzyme concentration, lower specific activity of an enzyme, or combinations thereof.

Many different methods can be used to produce a microorganism having reduced enzymatic and/or biological activity. For example, a microorganism can be engineered to have a genomic disruption in hemC and/or hemD using common mutagenesis or knock-out technology that renders the hemC and/or hemD sequence non-functional. Alternatively, antisense technology can be used to reduce enzymatic activity. For example, E. coli can be engineered to contain a cDNA that encodes an antisense molecule that prevents an enzyme from being made. The term “antisense molecule” encompasses any nucleic acid that contains sequences that correspond to the coding strand of an endogenous polypeptide. An antisense molecule also can have flanking sequences (e.g., regulatory sequences). Thus, antisense molecules can be ribozymes or antisense oligonucleotides. A ribozyme can have any general structure including, without limitation, hairpin, hammerhead, or axhead structures, provided the molecule cleaves RNA.

Exogenous nucleic acids can be introduced into microorganisms that have reduced activity. For example, an exogenous nucleic acid encoding HasR, an outer membrane receptor, can be introduced into a microorganism in which the endogenous hemC and hemD nucleic acid sequences are non-functional. See, Ghigo et al., J. Bacteriol. 179(11):3572-9 (1997) for a description of hasR. The resulting microorganism can have the ability to uptake hemin from the culture medium. In some embodiments, the exogenous nucleic acid also encodes a porphobilinogen deaminase polypeptide. In some embodiments, the porphobilinogen deaminase polypeptide is less than full-length (i.e., it is truncated). For example, the nucleic acid can encode one or more domains of the porphobilinogen deaminase polypeptide, such as the α and ω domains of the polypeptide. The exogenous nucleic acid further can encode a uroporphyrinogen III synthase.

Production of Porphyrins in Microorganisms

Typically, porphyrins are produced by culturing an engineered microorganism of the invention in culture medium then extracting the porphyrins from the cultured microorganisms or from the culture medium. In general, the culture media and/or culture conditions are such that the microorganisms grow to an adequate density and produce the porphyrin(s) efficiently. In some embodiments, porphyrin precursors such as succinate or glycine can be added to the medium to enhance porphyrin production. As described herein, production levels (up to 90 μM, roughly 50 mg/L) obtained with engineered microorganisms of the invention, without further optimization of cultivation conditions, is in the same order of magnitude as commercial production levels reported for vitamin B₁₂ (100-300 mg/L) using engineered microbial strains.

The invention will be further described in the following examples, which do not limit the scope of the invention described in the claims.

### EXAMPLES

In the following examples, for the sake of conciseness, the notation commonly adopted for operons is used to describe combinations of heme biosynthetic genes. Thus, hemAB means any combination of hemA and hemB, not necessarily an operon.

### Example 1

### High-Level Production of Porphyrins in Metabolically Engineered E. coli

A modular system was designed for the overproduction of porphyrins and porphyrinogens in E. coli. The system is based on three compatible replicons belonging to different incompatibility groups. Each of these plasmids carries one or several heme biosynthetic genes cloned from various bacteria (Table 1). All heme-specific genes in the pathways were constitutively expressed from a lac-promoter. Thus, regulation at the level of transcription and translation was (largely) avoided. Overproduction of a particular porphyrin (or porphyrinogen) can be achieved by choosing the appropriate combination of plasmids. This system was used to systematically extend the pathway (FIG. 1) and analyze the products formed. A three-plasmid system, with the appropriate “empty” control plasmids as necessary, was used throughout to assure similar growth conditions.

Bacterial Strains, Plasmids, and Growth Conditions

The strains and plasmids used in this example are listed in Table 1. E. coli JM109 was used as a host both for cloning and for assembly of the porphyrin/heme overproduction pathways. The plasmids pUCmod and pACmod (Schmidt-Dannert et al., 2000, Nat. Biotechnol. 18:750-3) and pBBR1MCS-2 (Kovach et al., 1995, Gene, 166:175-6), which confer carbenicillin, chloramphenicol, and kanamycin resistance, respectively, were used to construct porphyrin/heme biosynthetic pathways, as described below. Bacillus (B.) subtilis and E. coli were routinely grown in LB medium. For E. coli, the medium was supplemented with carbenicillin (100 μg/ml), chloramphenicol (50 μg/ml), and kanamycin (30 μg/ml) where appropriate. Rhodobacter (Rba.) capsulatus was grown in Rhodospirallacea medium as suggested by DSMZ and Synechocystis was grown in BG-11 medium as suggested by the ATCC.

TABLE 1
Plasmids and strains

| | | Source or |
|---|---|---|
| | Relevant properties or genotype | reference |
| Strains | | |
| E. coli (K12) JM109 | recA1 supE44 endA1 hsdR17 (rₖ⁻, mₖ⁺) gyrA96 | Yanish-Perron et al. |
| | relA1 thi Δ(lac-proAB) [F′, traD36 proAB⁺ lacI^{q} | (1985), Gene |
| | lacZΔM15] | 33: 103-19 |
| Rba capsulatus ATH 2.3.1 | Wildtype | DSMZ |
| Synechocystis sp. PCC6803 | Wildtype | ATCC |
| B. subtilis 168 | Wildtype | DSMZ |
| Plasmid | | |
| pUCMod | Cloning vector, constitutive lac-promotor, Amp^{R} | Schmidt-Dannert et |
| | | al., 2000, supra |
| pACMod | Cloning vector, Tc^{R}, Cm^{R} | Schmidt-Dannert et |
| | | al., 2000, supra |
| PBBR1MCS-2 | Cloning vector, Km^{R} | Kovach et al., 1995, |
| | | supra |
| pUCmod-hemA | hemA gene from Rba. capsulatus | This study |
| pUCmod-hemB | hemB gene from E. coli | This study |
| pUCmod-hemC | hemC gene from E. coli | This study |
| pUCmod-hemD | hemD gene from E. coli | This study |
| pUCmod-hemE | hemE gene from Synechocystis | This study |
| pUCmod-hemF | hemF gene from E. coli | This study |
| pUCmod-hemY | hemY gene from B. subtilis | This study |
| pUCmod-hemH | hemH gene from B. subtilis | This study |
| pACmod-hemA | Constitutively expressed Rba. capsulatus hemA | This study |
| pACmod-hemAB | Constitutively expressed Rba. capsulatus hemA and | This study |
| | E. coli hemB | |
| pACmod-hemABD | Constitutively expressed Rba. capsulatus hemA and | This study |
| | E. coli hemB and hemD | |
| pACmod-hemABC | Constitutively expressed Rba. capsulatus hemA and | This study |
| | E. coli hemB and hemC | |
| pACmod-hemABCD | Constitutively expressed Rba. capsulatus hemA and | This study |
| | E. coli hemB, hemB and hemC | |
| pBBR-hemE | Constitutively expressed Synechocystis hemE | This study |
| pBBR-hemEF | Constitutively expressed Synechocystis hemE and | This study |
| | E. coli hemF | |

DNA Manipulations Qiagen products (Hilden, Germany) were used for the isolation of genomic and plasmid DNA and to purify DNA fragments from agarose gels. Restriction enzymes, T4 DNA ligase and Vent DNA polymerase were from New England Biolabs (Beverly, Ma.). Klenow polymerase was from Takara (Shiga, Japan). Taq DNA polymerase in buffer A was from Promega (Madison, Wisc.). Oligonucleotide primers were from either Genemed Synthesis (South San Francisco, Calif.) or Integrated DNA Technologies (Coralville, Iowa). DNA sequencing was performed by the Advanced Genetic Analysis Center at the University of Minnesota.
Plasmid construction

Initial Cloning of Heme Biosynthetic Genes

Eight heme biosynthetic genes were amplified (using Taq DNA polymerase) from chromosomal DNA from Rba. capsulatus (hemA), E. coli (hemB, hem C, hemD and hemF), Synechocystis sp. (hemE) and B. subtilis (hem Y and hem H) (Table 1). The GenBank accession numbers of the genes are: X53309 (hemA), D85613 (hemB), X12614 (hemC and hemD), D64006 (hemE), X75413 (hemF) and M97208 (hemY and hemH). All genes were amplified from the corresponding genomic DNA using primers that had been designed to generate Xba I and Not I restriction sites, respectively, near the ends of the amplification products and a Shine-Dalgarno sequence upstream of the coding region. The PCR products were cloned into pUCmod, a derivative of pUC19 obtained by replacing a DNA stretch encompassing the lac-operator through the lacZα gene with a new multiple cloning site (Xba I, Sma I, EcoR I, Nco I and Not I) (see Schmidt-Dannert et al., 2000, supra). This vector facilitates constitutive expression of cloned genes when provided with a Shine-Dalgarno sequence.

Assembly of Heme Biosynthetic Genes on pACmod and pBBR1MCS-2

The cloned genes, with their respective lac-promoters, were reamplified (using Vent DNA polymerase) from the respective pUCmod plasmids with primers containing suitable restriction sites and assembled in pACmod (a pACYC184 derivative obtained by removing the Xba I site) or pBBR1MCS-2 as described below.

The hemA containing PCR product was subcloned into pACmod using Hind III (5′-end) and BamH I sites (3′-end) to construct pACmod-hemA. The hemB gene was amplified to produce a PCR product with a BamH I site (5′-end) and a Cla I site (3′-end). This PCR product was digested with only BamH I (leaving a blunt end at the other side) and ligated to the pACmod-hemA vector band that was prepared by digestion with Sal I, end-filling with Klenow and subsequent digestion with BamH I. This construction yielded pAC-hemAB; where hemA and hemB are contained on a ˜2.5 kb Cla I fragment. The BamH I site was then removed by digestion, end-filling and re-ligation, yielding pACmod-hemABΔBam. This procedure yields a new, third Cla I site that is protected by overlapping dam methylation. A PCR product carrying the hemD gene and restriction sites for BamH I (5′-end) and Xho I site (3′-end) was subcloned into BamH I and Sal I digested pACmod, resulting in pACmod-hemD. The ˜2.5 kb Cla I fragment from pACmod-hemABΔBam, encompassing the hemA and hemB genes, was subcloned into the Cla I site of pACmod-hemD to generate pACmod-hemABD.

Plasmid pACmod-hemABC was constructed in two stages. First, the hemC gene was subcloned as a Hind III (5′-end)-BamH I (3′-end) fragment into pACmod. Then the hemA and hemB containing Cla I fragment from pACmod-ABΔBam was cloned in the Cla I site. Plasmid pACmod-hemABCD was constructed by cloning the hemC gene as a BamH I fragment in pACmod-hemABD. The hemE gene was subcloned into the XhoI and Hind III sites of pBBR1MCS-2. Similarly, the hemF gene was subcloned into the EcoR I and BamH I sites of pBBR1-MCS2 to construct pBBR1-hemF. The plasmid pBBR1-hemEF was constructed by subcloning the hemF gene into pBBR1-hemE with the EcoR I and BamH I sites.

Overproduction and Analysis of Porphyrinogens and Porphyrins

Various combinations of plasmids were transformed into E. coli. A three-plasmid system was used throughout to ensure similar culture conditions. All transformants were cultivated for 48 hrs at 30 C (to lower the copy number of pUCmod (Lin-Chao et al., 1992, Mol. Microbiol. 6:3385-93) in 50 ml of LB medium containing carbenicillin (100 μg/ml), chloramphenicol (50 μg/ml), and kanamycin (30 μg/ml). Each culture was processed as follows. After cultivation, 200 mg of DEAE-Sephadex A-25 (Pharmacia Fine Chemicals, Uppsala, Sweden) was added to the culture to adsorb secreted or released porphyrin(ogen)s (Olsson et al., 2002, J. Bacteriol. 184:4018-24). The bacterial cells and the DEAE resin were pelleted by centrifugation (4,000×g, 20 minutes). The pellet was extracted with 50 mL of organic solvents (acetone:dimethyl formamide:6 N HCl (10:1:2=v:v)) at 40 C. During this procedure, all porphyrinogens were converted to the corresponding porphyrins by contact with atmospheric oxygen. Fifty μL of the extract was applied to an ODS column (Zorbax RX-C18, 150×4.6 mm, 5 μm; Agilent Technologies, Palo Alto, Calif.) using an Agilent HPLC system equipped with a photodiode array detector (PDA). A previously described mobile phase system was used (Ng et al., 2002, Toxicol. Lett. 133:93-101) with minor modifications. Briefly, the mobile phase consisted of two solvent mixtures, solvent A (100 g/l ammonium acetate and 63 mL of acetonitrile, adjusted to pH 5.16 with glacial acetic acid) and solvent B (methanol:acetic acid (10:1=v:v)). Analytes were eluted at a flow rate of 1 mL/min. The program used consisted of an isocratic elution step with solvent A (5 min) followed by a linear gradient from 0 to 100 % solvent B in 30 min. Solvent B was then maintained at 100% for 5 min. The system was equilibrated for another 5 min at 100% of solvent A before the next sample injection. The mass spectra of various porphyrins and heme were obtained using MS (ThermoFinnigan's LCQ, SanJose, Calif.) coupled with electrospray ionization (ESI) interface. The spray voltage was 4.0 kV and the heated capillary temperature was 225° C. The amounts of individual porphyrins and heme were quantified using authentic porphyrins and heme purchased from Porphyrin Products Inc. (Logan, Utah) and ICN Biomedicals Inc. (Aurora, Ohio), respectively.

B. subtilis ferrochelatase activity was assayed essentially as described in Camadro and Labbe (1988), J. Biol. Chem. 263:11675-82, except that the palmitic acid and Tween 80 were omitted as B. subtilis ferrochelatase is a soluble enzyme. Cell-free extracts were made from three assembled pathways: (1) cells with hemA through hemH (combination 10 in Table 2), (2) cells with hemA through hemF (combination 9 in Table 2) and (3) cells with no overexpressed heme biosynthetic genes (combination 1 in Table 2). The latter two cell-free extracts served as (negative) controls. Reaction buffer without cell-free extract served as a third control. All transformants were grown for 48 hr at 30° C., harvested by centrifugation (4000×g, 15 min.) and resuspended in 50 mM Tris-HCl buffer (pH 7.6). The cells were disrupted by sonication. Ferrochelatase activity was monitored by time-dependent measurement of fluorescence at room temperature (excitation at 410 nm and emission at 632 nm) using a Gemini XS spectrofluorometer from Molecular Devices (Sunnyvale, Calif.).

Results

Porphyrin(ogen) Overproduction

E. coli transformants overexpressing Rba. capsulatus hemA developed a slightly brown color compared to control cells after overnight's growth at 30° C., both on LB-agar plates and in liquid culture due to the accumulation of porphyrins. However, a strong reddish color was only observed when the first three genes (hemA, hemB, hemC) were over-expressed together. Furthermore, the coloration and its intensity depended strongly on the type of porphyrin produced, and color continued to develop after cells had reached the stationary phase. E. coli cells overexpressing at least the first five genes of the assembled heme pathway (hemA-E) (FIG. 1), accumulated large amounts of porphyrins in the culture medium resulting in partial precipitation of porphyrins (brown porphyrin precipitate). The porphyrin(ogen) content from liquid cultures of the various transformants was analyzed by HPLC and mass spectrometry. During sample preparation all porphyrinogens were converted to the corresponding porphyrins by contact with atmospheric oxygen, as no porphyrinogens were detected by mass spectrometry.

Systematic Extension of an Engineered Heme Biosynthetic Pathway

Tetrapyrrole production starts with the biosynthesis of the precursor 5-aminolevulinic acid (ALA). Two routes for ALA synthesis are known: from succinyl-CoA and glycine (the C₄ pathway) and from tRNA^{Glu} (the C₅ pathway). The C₄-pathway is found in animals, fungi, non-photosynthetic eukaryotes and in the α-proteobacteria. Most bacteria (including E. coli) and plants use the C5 pathway. The C4 route was used for ALA biosynthesis in the pathway depicted in FIG. 1 for the following reasons. Only one heme specific gene, hemA, is required for ALA synthesis using the C4 pathway, while two genes (gtrA and hemL) are needed in the C5 pathway. (The designation gtrA in the C5 pathway replaces the former designation hemA, as the hemA genes of the C4 and the C5 pathway encode unrelated enzymes.) In addition, the use of tRNA^{Glu} for synthesizing ALA has been found to have an adverse effect on protein synthesis.

Overexpression of the Rba. capsulatus hemA gene in recombinant E. coli cells resulted in the overproduction of porphyrins (Table 2) due to conversion of ALA by chromosomally encoded heme biosynthetic enzymes.

The E. coli enzyme 5-aminolevulinic acid dehydratase, also known as porphobilinogen synthase (encoded by hemB), is the second enzyme in our engineered pathway. It converts two ALA molecules to porphobilinogen (PBG) with the concomitant release of a water molecule. Co-expression of hemA and hemB did not result in a higher porphyrin production than overexpression of hemA alone (cf. combinations 2 and 3, Table 2).

TABLE 2
Porphyrin accumulation in E. coli transformants overexpressing different combinations of heme biosynthetic genes^{a}.

| | pBBR1- | | Uro^{b} (μmol/L) | | 5-Carboxy | Copro (μmol/L) | | Proto | Heme | Total |
|---|---|---|---|---|---|---|---|---|---|---|
| pACmod | MCS2 | pUCmod | I | III | (μmol/L) | I | III | (μmol/L) | (μmol/L) | (μmol/L) |
| — | — | — | n.d.^{d} | n.d. | n.d. | n.d. | n.d. | trace^{f} | 0.2 ± 0.0 | 0.2 ± 0.0 |
| A^{c} | — | — | 3.3 ± 0.8^{e} | 0.6 ± 0.2 | n.d. | 0.3 ± 0.0 | 2.0 ± 0.1 | 0.6 ± 0.5 | 0.9 ± 0.1 | 7.7 ± 1.6 |
| A + B | — | — | 4.0 ± 1.8 | 0.6 ± 0.3 | n.d. | 0.1 ± 0.0 | 0.9 ± 0.2 | 0.6 ± 0.1 | 0.4 ± 0.1 | 6.7 ± 2.4 |
| A + B + D | — | — | 0.8 ± 0.1 | 1.8 ± 0.1 | n.d. | 0.1 ± 0.0 | 1.0 ± 0.3 | 1.1 ± 0.1 | 0.5 ± 0.1 | 5.3 ± 0.7 |
| A + B + C | — | — | 14.4 ± 0.2 | n.d. | 1.1 ± 0.2 | 0.5 ± 0.1 | n.d. | Trace | 0.2 ± 0.1 | 16.2 ± 0.5 |
| A + B + C + D | — | — | 7.2 ± 1.8 | 41 ± 10 | n.d. | n.d. | 1.6 ± 0.3 | 0.2 ± 0.1 | 0.4 ± 0.3 | 49 ± 12 |
| A + B + C | E | — | 8.2 ± 0.1 | n.d. | 2.2 ± 0.1 | 1.0 ± 0.1 | 0.2 ± 0.0 | Trace | 0.2 ± 0.1 | 11.7 ± 0.4 |
| A + B + C + D | E | — | n.d. | n.d. | n.d. | 3.0 ± 0.2 | 47 ± 4 | 0.4 ± 0.2 | 0.4 ± 0.1 | 51 ± 4 |
| A + B + C + D | E + F | — | n.d. | n.d. | n.d. | 0.3 ± 0.1 | 0.7 ± 0.2 | 82 ± 6 | 2.4 ± 0.5 | 85 ± 7 |
| A + B + C + D | E + F | H | n.d. | n.d. | n.d. | 0.3 ± 0.1 | 1.0 ± 0.2 | 84 ± 8 | 3.3 ± 0.3 | 89 ± 9 |
| A + B + C + D | — | Y | 2.0 ± 0.2 | 7.2 ± 0.6 | n.d. | n.d. | 1.4 ± 0.3 | Trace | 0.1 ± 0.1 | 11 ± 1 |
| A + B + C + D | F | Y | 0.7 ± 0.1 | 4.8 ± 0.1 | n.d. | n.d. | 2.6 ± 0.1 | 0.5 ± 0.1 | 0.3 ± 0.1 | 9.3 ± 0.4 |
| A + B + C + D | E | Y | 1.3 ± 0.3 | 0.2 ± 0.1 | n.d. | n.d. | 37 ± 2 | Trace | 0.7 ± 0.0 | 39 ± 2 |
| A + B + C | E | Y | 7.0 ± 0.7 | n.d. | 4.7 ± 0.6 | 2.1 ± 0.4 | 1.8 ± 0.3 | n.d. | 0.2 ± 0.1 | 16 ± 2 |
| A + B + C + D | E + F | Y | 0.7 ± 0.3 | 0.4 ± 0.1 | n.d. | 1.5 ± 0.2 | 21 ± 3 | 28 ± 4 | 1.7 ± 0.4 | 52 ± 8 |

^{a}E. coli cells were cultured in LB media containing chloramphenicol, kanamycin, and carbenicillin for 48 h at 30° C.;
^{b}abbreviations: uro: uroporphyrin; 5-carboxy: pentacarboxyporphyrin; copro: coproporphyrin; proto: protoporphyrin;
^{c}heme biosynthetic genes overexpressed on pACmod, pBBR1-MCS2, pUCmod;
^{d}n.d. means not detected;
^{e}values are averages of triplicate experiments and are given as mean ± standard deviation;
^{f}trace: less than 0.1 μmol/L

The enzyme 1-hydroxymethylbilane synthase (or porphobilinogen deaminase), encoded by hemC, catalyzes the tetramerization of porpobilinogen to the linear product 1-hydroxymethylbilane (HMB), the immediate precursor of the uroporphyrinogens (uro'gens) I and III. The physiologically relevant III isomer is formed in the presence of the next enzyme of the heme biosynthetic pathway, uro'gen III synthase (encoded by hemD). Co-overexpression of hemA through hemD results in the production of mainly the III isomer. In the absence of uro'gen III synthase spontaneous cyclization of 1-hydroxymethylbilane leads to the formation of the formation of the I isomer. Overexpression of hemA, hemB and hemC led to formation of mainly the I isomer (Table 2), despite the presence of the chromosomal hemD gene.

The next enzyme in the pathway of FIG. 1, uro'gen decarboxylase (encoded by hemE), is derived from Synechocystis sp. This enzyme accepts both uro'gen I and uro'gen III as substrates and converts them to the corresponding coproporhyrinogens (copro'gens). A significant amount of an intermediate with five carboxyl groups accumulated as a result of incomplete uro'gen I decarboxylation (Table 2).

Copro'gen III is converted to protoporphyrinogen (proto'gen) IX by the action of a copro'gen III oxidase (CPO). The oxygen-dependent enzyme in our pathway is encoded by the E. coli hemF gene. When overexpressed, as in our pathway, the enzyme further oxidizes proto'gen IX to the fully conjugated product protoporphyrin IX. The enzyme does not use copro'gen I as a substrate (Table 2).

Although no enzyme is required for the conversion of proto'gen IX to protoporphyrin IX in our pathway (as this reaction is catalyzed by CPO when the enzyme is overproduced), we have incorporated the enzyme proto'gen IX oxidase (PPO) from B. subtilis (encoded by hem Y). This enzyme has the peculiar property not only to convert proto'gen IX to protoporphyrin IX, but also to convert copro'gen III to coproporphyrin III. Incorporation of this enzyme in the assembled pathway resulted in a competition of the copro'gen III oxidase and the proto'gen IX oxidase for the substrate copro'gen III, thus leading to the accumulation of both coproporphyrin III and protoporphyrin IX (cf. combinations 9 and 15 in Table 2). The accumulation of coproporphyrin III is remarkable, as it is not considered to be an intermediate in natural porphyrin synthesis (in contrast to copro'gen III).

The last enzyme in the pathway is the ferrochelatase from B. subtilis (the hemH gene product). The enzyme from B. subtilis was chosen since its crystal structure has been determined. Ferrochelatase incorporates a ferrous iron ion into protoporphyrin IX, yielding protoheme IX (heme) (Table 2).

Production Levels

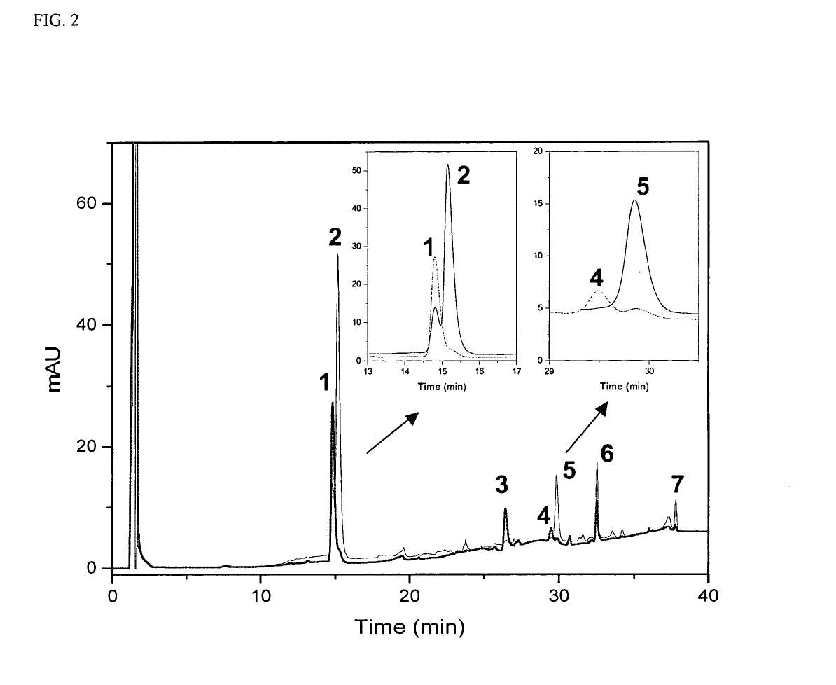

The porphyrin production resulting from various combinations of plasmids is presented in Table 2. All E. coli transformants listed in Table 2 grew to similar optical densities (600 nm) of about 3.5 after 48 hrs cultivation at 30° C. regardless of the overexpressed heme genes. Porphyrin peaks were identified by their spectroscopic properties, molecular weight and relative retention times on HPLC. Two typical HPLC profiles are shown in FIG. 2. The separated porphyrins in FIG. 2 were identified as uroporphyrin I (peak 1, λₘₐₓ=503 537 567 618 nm; [M+H]⁺ at m/z=831.4), uroporphyrin III (peak 2, λₘₐₓ=503 537 567 618 nm; [M+H]⁺ at m/z=831.4), 5-carboxyporphyrin (peak 3, λₘₐₓ=504 534 565 617 nm; [M+H]⁺ at m/z=699.4), coproporphyrin I (peak 4, λₘₐₓ=498 532 566 618 nm; [M+H]⁺ at m/z=655.4), coproporphyrin III (peak 5, λₘₐₓ=498 532 566 618 nm; [M+H]⁺ at m/z=655.4), heme (peak 6, λₘₐₓ=502(α), 628(β), 536(shoulder) nm; [M]⁺ at m/z=616.3), protoporphyrin IX (peak 7, λmax=505 539 574 628 nm; [M+H]⁺ at m/z=563.4).

In most cases, the major porphyrin present in the samples was the one formed by the action of the last overexpressed gene in the pathway. In many cases, small amounts of other porphyrins were also present due to the activities of chromosomally encoded heme biosynthetic enzymes, e.g., gene combinations 1-6. The total production of porphyrins varied considerably, from 5 to almost 90 μM, but was always considerably higher in the assembled overproduction pathway than in the control (E. coli JM109 carrying plasmids without heme biosynthetic genes) (Table 2).

The overexpression of hemA leads to a significant (nearly 400-fold) increase in the amount of porphyrins formed. Co-overexpression of hemB did not further enhance porphyrin production, but introduction of the hemC and hemD genes again boosted the production of porphyrins an additional 2 to 7 times. In all cases, however, the total porphyrin production was lower in combinations with the pACmod-hemABC plasmid than with the pACmod-hemABCD plasmid. This is, at least in part, due to a lower stability of the former plasmid. For both plasmids, some white colonies always appeared on plates, amidst a majority of orange-red colonies. Relatively more white colonies were seen for the pACmod-hemABC plasmid. Analysis of plasmid DNA of white colonies (not shown) showed both truncated plasmids and intact plasmids that apparently had one or more inactivated genes.

Gene combinations in which the plasmids pACmod-hemABCD and pUCmod-hem Y were combined (combinations 11, 12, 13 and 15 in Table 2) produced drastically less porphyrins (about 30-40 %) than combinations without the latter plasmid (combinations 6, 8, 9, 10 in Table 2). This effect appeared to be more profound in the absence of hemE (combinations 11 and 12 versus 6 in Table 2). No adverse effects on the E. coli cells were observed when the hemY gene was constitutively expressed from a lac-promoter on a high copy number plasmid (the pUC19 derivative pUCmod).

Combination of the plasmids pACmod-hemABCD and pUCmod-hemE resulted in the formation of large quantities of coproporphyrin III. Uroporphyrin III is no longer detected. The total amount of porphyrins formed appeared not to be affected by the presence of uro'gen III decarboxylase (cf. combinations 6 and 8, Table 2). In contrast, when the plasmids pACmod-hemABC and pUCmod-hemE were combined, uroporphyrin I was still the main product, although smaller amounts of coproporphyrin I and pentacarboxyporphyrin were formed as well, reflecting the lower activity of the enzyme towards uro'gen I (Table 2).

Co-expression of hemF, coding for the E. coli CPO, resulted in a 70-80 % further increase in porphyrin production (combination 9 and 10 versus 8 in Table 2). The major product, protoporphyrin IX, was largely released into the medium where it precipitated.

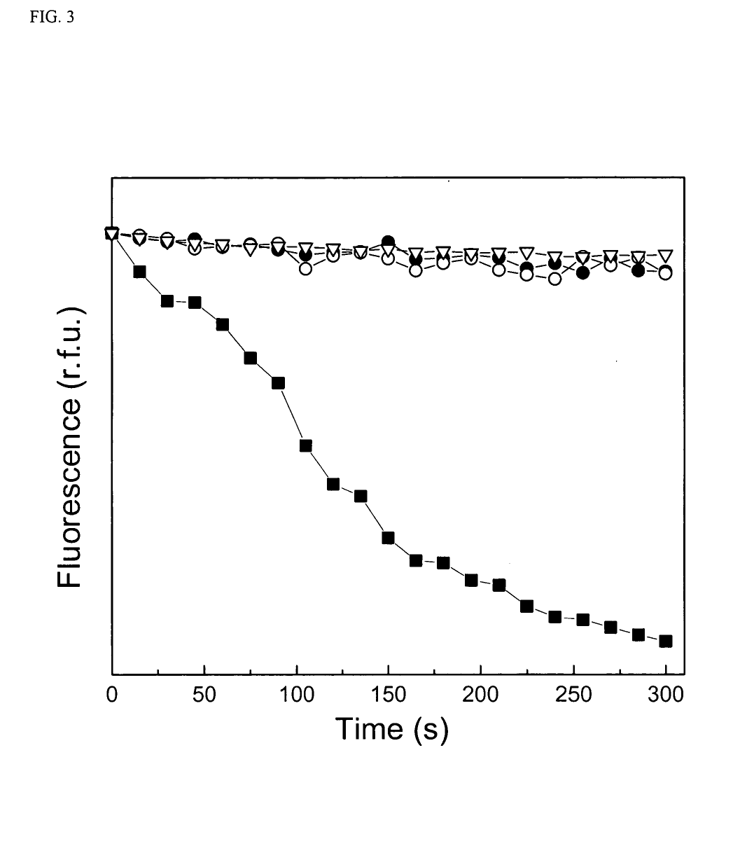

Surprisingly, presence of the ferrochelatase (product of hemH) in the pathway did not lead to a significant accumulation of heme (Table 2), although the hemH gene was highly expressed and that ferrochelatase activity could be demonstrated in vitro (FIG. 3) using a lysate of an E. coli transformant harboring plasmids pACmod-hemABCD, pBBR1-hemEhemF and pUCmod-hemH.

### Example 2

### Construction of a Hemin-Permeable Strain, a ΔhemCD Strain and Splitting and Truncation of hemC

Typically, E. coli K12 strains are not hemin-permeable, but spontaneous hemin-permeable strains have been obtained from strains defective in heme biosynthesis. A hemin-permeable derivative of E. coli JM109 was constructed by integrating the Serratia marcescens hasR gene in its chromosome, using the system devised by Haldiman and Wanner (J. Bacteriol. 183(21):6384-6393 (2001)). Bacteria and plasmids used in this example are listed in Table 3. The encoded protein, HasR, is an outer membrane receptor that is part of the Serratia marcescens iron acquisition system. Heterologous expression of hasR enables E. coli to take up hemin from the growth medium (Ghigo et al., J. Bacteriol. 179(11):3572-9 (1997)). Plasmid pFR2 containing the hasR gene under control of the arabinose-inducible P_{araB} promoter was a gift from Prof. Wandersman (Institut Pasteur, Paris, France). The plasmid was digested with NsiI and XmnI and the fragment with the hasR gene and the P_{araB} promoter and its regulatory sequences was ligated into pLA2 digested with PstI and SmaI. The ligation mixture was transformed into BW25142. The construct, pLA2-hasR was verified by restriction analysis and transformed into JM109 harboring helper plasmid pINT-ts. A few transformants were obtained by selection for kanamycin resistance and analyzed by PCR as suggested by Haldiman and Wanner. At least two copies of pLA2-hasR were present in the chromosome of each of the transformants resulting in the heme-permeable strain: E. coli ADB=JM109 attl::pLA-hasR (araC+hasR+KmR). One integrant, named ADB1, was selected for further use.

TABLE 3
STRAINS AND PLASMIDS

| Strain or Plasmid | Relevant genotype/properties | Source or reference |
|---|---|---|
| Strains | | |
| JM109 | recA1 supE44 endA1 hsdR17 (rₖ⁻, mₖ⁺) gyrA96 relA1 thi Δ(lac- | Yanisch-Perron, 1985 |
| | proAB) [F′, traD36 proAB⁺ lacI^{q} lacZΔM15] | |
| ADB1 | (JM109) attB_{λ}::pLA2-hasR (Km^{R}); hemin uptake under | This study |
| | arabinose control | |
| ADB7 | (JM109) attB_{λ}::pLA2-hasR (Km^{R}) ΔhemCD::cat (Cm^{R}) | This study |
| ADB8 | Spontaneous hemin-permeable derivative of ADB7 (arabinose- | This study |
| | independent) | |
| ADB9 | Derivative of ADB8 obtained by excision of pLA2-hasR (Km^{s}) | This study |
| BW25142 | uidA::pir-116 recA; host for conditional plasmids carrying oriR_{γ} | Haldimann, 2001 |
| Plasmids | | |
| pLA2 | oriR_{γ} kan attP_{λ}; Vector for integration in chromosome at attB_{λ} | Haldimann, 2001 |
| pFR2 | S. marcescens hasR gene cloned in pBAD24 (under control of | Prof. Wandersman |
| | P_{araB}) | |
| pLA2-hasR | S. marcescens hasR gene and arabinose promoter region from | This study |
| | pFR2 cloned in pLA2 | |
| pINT-ts | OriR101 repA101ts int_{λ} bla; helper plasmid for integration | Haldimann, 2001 |
| pAH57 | OriR101 repA101ts int_{λ} xis_{λ} bla; helper plasmid for integration | Haldimann, 2001 |
| pKD3 | oriR_{γ} cat bla; template plasmid for gene disruption | Datsenko, 2000 |
| pKD46 | oriR101 repA101ts γ β exo bla; helper plasmid for gene | Datsenko, 2000 |
| | disruption | |
| pACmod-hemAB | constitutive expression of hemA and hemB | Kwon, 2003 |
| pUCmod-hemC | constitutive expression of hemC from E. coli | [Kwon, 2003 |
| pUCmod-hemD | constitutive expression of hemD from E. coli | [Kwon, 2003 |
| pUC19-hemD | constitutive expression of hemD, SalI site in hemD removed | This study |
| pUC19-hemChemD | constitutive expression of hemC and hemD | This study |
| pUC19 | general cloning vector | Yanisch-Perron, 1985 |
| pUCmod | Cloning vector, constitutive lac-promotor, Amp^{R} | Schmidt-Dannert, |
| | | 2000 |
| pSPLIT101-hemD | split hemC; hemD | This study |
| pSPLIT117-hemD | split hemC; hemD | This study |
| pSPLIT196-hemD | split hemC; hemD | This study |
| pSPLIT208-hemD | split hemC; hemD | This study |
| pSPLIT218-hemD | split hemC; hemD | This study |
| pSPLIT241-hemD | split hemC; hemD | This study |
| pSPLIT242-hemD | split hemC; hemD | This study |
| pTRUNC101-hemD | truncated hemC; hemD | This study |
| pTRUNC117-hemD | truncated hemC; hemD | This study |
| pTRUNC196-hemD | truncated hemC; hemD | This study |
| pTRUNC208-hemD | truncated hemC; hemD | This study |
| pTRUNC218-hemD | truncated hemC; hemD | This study |
| pTRUNC241-hemD | truncated hemC; hemD | This study |
| pTRUNC242-hemD | truncated hemC; hemD | This study |

A heme-permeable and hemC, hemD deletion strain of E. coli was created by deleting hemCD from ABD1 by the method of Datsenko and Wanner (Proc. Natl. Acad. Sci. USA (2000) 97(12):6640-5). Briefly, a PCR product was generated that contains a chloramphenicol resistance gene flanked by regions that are homologous to chromosomal DNA flanking the hemCD genes. This PCR product was obtained by amplification of the chloramphenicol resistance gene from template plasmid pKD3 and two primers that anneal to the template and have long tails that provide the homology to the hemCD flanking sequences. The PAGE-purified primers had the following sequences: ΔhemCD-F (homology upstream of hemCD): 5′-GGATGTTAGGATGGACCACGGATGATAATGACGGTAACAAGCGTGTAGGCTGGAGC TGCTTC-3′ (62 nt, SEQ ID NO:1); ΔhemCD-R (homology downstream of hemCD): 5′-CCTGGTCTCTTCAACCACGGCGGAGGTTTTTTCTTGTTCCGTCATATGAATATCCTCC TTAG-3′ (62 nt, (SEQ ID NO:2).

The PCR product was transformed into ADB1 harboring pKD46 as suggested (Datsenko and Wanner). Transformants were selected on LB-agar plates containing chloramphenicol, hemin, and arabinose. After 48 hours at 37° C. a few colonies were obtained. These were tested for hemin-auxotrophy by restreaking them on plates with and without hemin and arabinose. One colony had the desired phenotype. In order to verify that the new phenotype resulted from the expected recombination between the PCR product and the chromosome, chromosomal DNA was isolated and digested with BamHI and PstI. Fragments of approximately in the range of 1.5 to 3 kb were gel-purified and ligated into pUC19 digested with the same enzymes. The ligation mixture was then used to transform JM109 to chloramphenicol resistance. Plasmid mini preparations from a few of the resulting colonies revealed an insert of 1.6 kb (the smallest possible fragment BamHI-PstI fragment that contains the chloramphenicol resistance gene and its flanking sequences). DNA sequence analysis revealed that both expected crossovers had occurred without aberrations. The new strain was named ADB7. This strain retained the F′ plasmid present in the parent strain JM109 as evidenced by the appearance of blue colonies after transformation of ADB7 with pUC19 and growth on LB-agar plates containing ampicillin, X-gal, IPTG, hemin and arabinose.

To test complementation of hemCD deletion, ADB7 was transformed with pUC19, pUCmod-hemC, and pUC19-hemChemD. The transformation mixture was plated on LB-agar containing ampicillin, but no hemin and arabinose. After overnight's growth at 37° C. normal-sized (like JM109) colonies were observed for pUC19-hemChemD. No growth was observed for either pUC19 or pUCmod-hemC.

As mentioned, ADB7 needs both hemin and arabinose for growth. Colony size on agar plates containing arabinose and hemin varied considerably under all conditions tested, i.e., 1-10 mM arabinose and 10⁻⁶-10⁻⁴ M hemin. This may be related to the facts that JM109 can metabolize arabinose and lacks the low-affinity high-capacity AraE transporter. Non-stochastic expression of genes under control of the P_{araB} promoter has been reported frequently. A spontaneous mutant of ADB7 that does not require arabinose for hemin uptake was isolated by restreaking ADB7 on LB agar with hemin but without arabinose. Colonies formed by this mutant, ADB8, have a uniform colony size and have a mucous appearance. Spontaneous hemin permeable mutants have been obtained frequently. The origin of the mutation is unknown. It is likely that the mutation in ADB8 is not in the hasR gene or its regulatory sequences, as the integrated plasmid pLA2-hasR could be excised (yielding ADB9) without affecting hemin uptake.

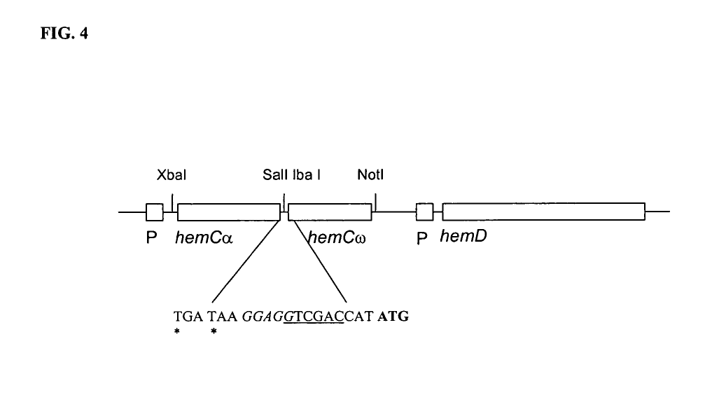

A series of hemC mutants was made by splitting the gene at different positions into two genes, yielding the pSPLIT series. The splitting (and truncation) points were chosen in loop regions of the enzyme (Louie et al., Proteins (2000) 25(1):48-78) after the residues R101, D117, V196, D208, A218, G241 and C242. Thus, the pSPLIT series was created by dividing the hemC gene into two new genes, hemCα and hemCω. This was done by the insertion of two stop codons after the first part of the gene, followed by a Shine-Dalgarno sequence, a SalI restriction site and a start (methionine) codon in frame with the remainder of the hemC gene (FIG. 4). The XbaI-NotI fragment containing the split hemC genes was subsequently exchanged with the corresponding fragment in pUC19-hemChemD (FIG. 4). The latter plasmid was constructed as follows. First, the XbaI and NotI sites in pUCmod-hemD were removed by digestion with the respective restriction enzymes followed by end-filling with Klenow and re-ligation. The hemD gene and the promoter region then were amplified with primers containing BamHI and HindIII sites and ligated into pUC19. The hemC gene was amplified from pUCmod-hemC and ligated in pUC19-hemD. Finally, a silent mutation was introduced to remove the SalI site in the hemD gene. The pTRUNC series was obtained from the pSPLIT series by deletion of the second part of the split hemC genes, hemCω (by digestion with SalI and NotI followed by end-filling and re-ligation).

Plasmid pUC19-hemChemD and the derivatives with split (resulting in the expression of an α- and ω-fragment of hemC) or truncated hemC genes (Δω) were introduced in ADB7 and plated on LB-agar with ampicillin and chloramphenicol but without hemin, to test the ability of the various constructs to complement ADB7 to autotrophic growth. Both pUCmod-hemC and pUC19 were included as negative controls. Growth at 37° C. was allowed for ˜40 hours and checked periodically. As already was established above, normal, wildtype-like growth, was observed after ˜18 hrs at 37° C. when ADB7 (or ADB8) was complemented with pUC19-hemChemD, but not with either pUC19 or pUCmod-hemC. Surprisingly, almost all of the plasmids with split or truncated hemC genes could restore autotrophic growth, although growth rates varied considerably (as judged by the order of the appearance and size of colonies on agar plates (Table 4). Growth is given as observed colony size (on a scale from 0 (no growth) to 6 (maximal growth)) after 37 h incubation at 37° C. on LB plates. The positions of the dissections and truncations are indicated in the ribbon structure of hemC (FIG. 5). However, none of these constructs restored wild-type growth rates. No growth was observed for the negative control pUC19 even after 40 hours. Surprisingly, some growth occurred when ADB7 was complemented with pUCmod-hemC, in the absence of hemD. Thus, E. coli may have an unidentified protein that can convert 1-hydroxymethylbilane into uro'gen III.

An analysis of genome sequences for heme biosynthetic enzymes has revealed that no hemD gene is present in several microorganisms that are nevertheless are able to synthesize heme, suggesting that another type of enzyme may exist for uro'gen III synthesis. Alternatively, some uro'gen III may form non-enzymatically from 1-hydroxymethylbilane or by isomerization from uro'gen I. It is known that all four uro'gen isomers can be formed from porphobilinogen in the absence of enzymes, and that those isomers can be interconverted under acidic conditions. Apparently, not enough uro'gen III is formed when both hemC and hemD are absent, as no growth was observed for the ADB7 containing “empty” pUC19.

TABLE 4

| Plasmid | Growth (ADB7 or ADB8)^{a} |
|---|---|
| pSPLIT-101 | 4 |
| pSPLIT-117 | 4 |
| pSPLIT-196 | 0 |
| pSPLIT-208 | 3 |
| pSPLIT-218 | 3 |
| pSPLIT-241 | 5 |
| pSPLIT-242 | 3 |
| pTRUNC-101 | 1 |
| pTRUNC-117 | 1 |
| pTRUNC-196 | 2 |
| pTRUNC-208 | 1 |
| pTRUNC-218 | 0 |
| pTRUNC-241 | 2 |
| pTRUNC-242 | 3 |
| pUC19-hemChemD^{b} | 6 |
| pUC19-hemD^{c} | 0 |
| pUCmod^{c} | 0 |
| pUCmod-hemC^{c} | 2 |
| ^{a}Growth of the ΔhemCD strains ADB7 and ADB8 when transformed with the indicated plasmids and plated on LB-agar plates with ampicillin (but without hemin). Growth was observed periodically during at least 48 hrs at 37° C. The experiment was performed twice using ADB7 as a host and once using ADB8 as a host. | |
| # The positive control was the first to appear, no colonies where visible for the negative controls. | |

^{b}Positive control (wildtype hemC and hemD genes)
^{c}Negative controls (no hemC)

### Example 3

### Improvement of Metalloporphyrin (Heme) Biosynthesis in E. coli

High-yield production of protoporphyrin IX in E. coli was achieved by overexpressing the hemABCDEFH genes. However, protoporphyrin IX was not efficiently converted to metalloporphyrin (heme) in vivo even though ferrochelatase (hemH) was overexpressed in E. coli and shown to be active. One possible reason for the accumulation of protoporphyrin IX could be insufficient metal availability in the cell due to tightly regulated metal transport in response to the intracellular metalloporphyrin (heme) concentration. To overcome this bottleneck, the ZupT metal uptake system was cloned and overexpressed in E. coli together with the hemABCDEFH genes.

Cloning of ygiE.

Recently, the ygiE gene (zupT) from E. coli was characterized as broad-range metal ion transporter (Grass et al, 2002 J. Bacteriol. 184:864-66). Thus, the ygiE gene (GenBank Accession No. AE000386) was amplified from chromosomal DNA of E. coli by using the following PCR primers, which were designed to generate XbaI and NotI restriction sites, respectively, and a Shine-Dalgarno sequence upstream of the coding region:
5′-GCTCTAGAAGGAGGATTACAAAATGTCAGTACCTCTCATTC-3′ (SEQ ID NO:3) and 5′-TAAAGCGGCCGCTTAACCAATTCCCGCCGTTTG-3′ (SEQ ID NO:4).

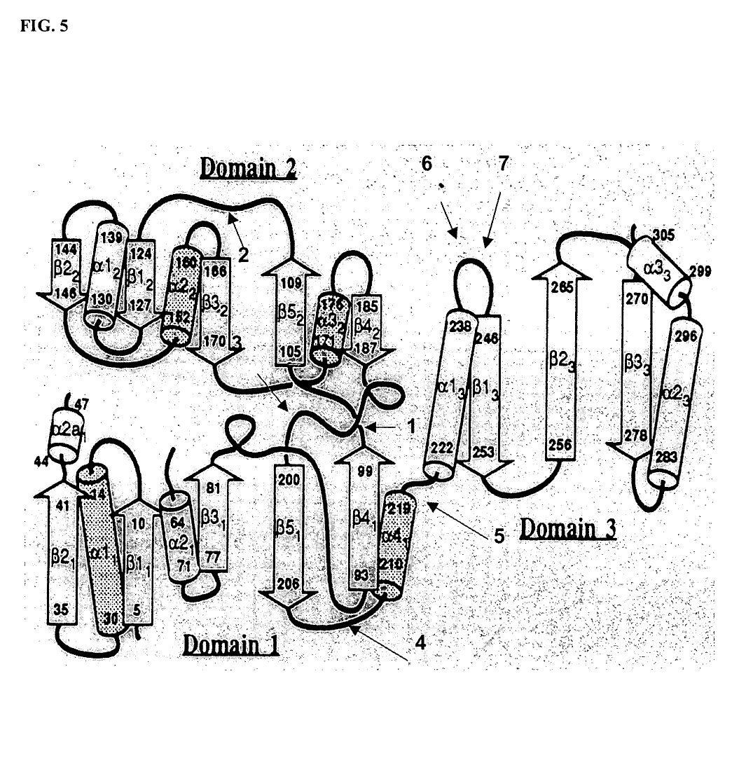

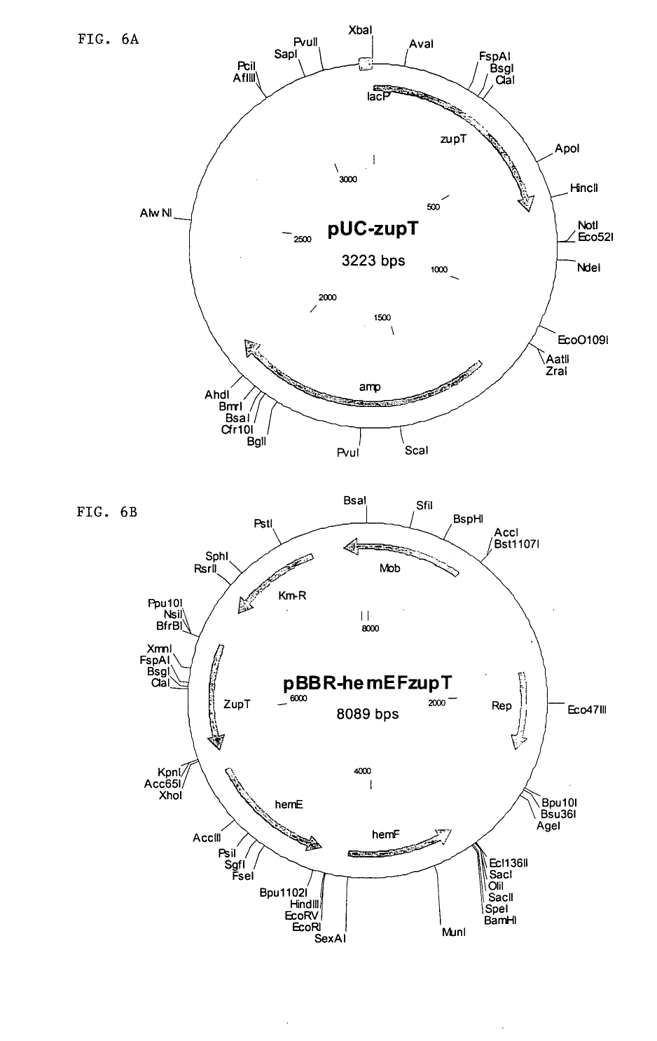

The PCR product was cloned into pUCmod, a derivative of pUC19, which facilitates constitutive expression, resulting in the construction of pUC-zupT (FIG. 6A). The cloned gene (ygiE) was reamplified from pUC-zupT with primers containing NsiI and KpnI sites. The PCR product was assembled into pBBR1-hemEF resulting in the construction of pBBR-hemEFzupT (FIG. 6B).

Analysis of Metalloporphyrins.

E. coli transformants were cultivated for 24 hrs at 30° C. in 50 mL of LB media containing carbenicillin (100 μg/ml), chloramphenicol (50 μg/ml), and kanamycin (30 μg/ml). After 24 hrs, 100 μM of ZnSO₄ and FeSO₄ were added into the media, respectively, and the bacterial cells were cultivated again for another 24 hrs. When FeSO₄ was added into the media, the cells were grown anaerobically to prevent oxidation of the ferrous ion. After 48 hrs, the cells were harvested by centrifugation (4,000×g, 20 minutes). The cell pellets were suspended in a 50 mM EDTA solution in 100 mM of Tris-HCl buffer (pH 7.6) to remove the divalent ions added. The cells were then washed twice with Tris-HCl buffer (pH 7.6) and subsequently extracted with acetone for Zn-proto IX analysis and with acetone hydrochloric acid solution (acetone: 6 N HCl=10:1) for heme analysis, respectively. The extracts were applied to an ODS column (Zorbax RX-C18, 150×4.6 mm, 5 μm; Agilent Technologies, Palo Alto, Calif.) using an Agilent HPLC system equipped with a photodiode array detector (PDA). The mobile phase consisted of two solvent mixtures, solvent A (100 g/l ammonium acetate and 125 mL of acetonitrile, adjusted to pH 5.16 with glacial acetic acid) and solvent B (methanol:acetic acid (10:1=v:v)). Analytes were eluted at a flow rate of 1 mL/min. The program used consisted of an isocratic elution step with solvent A (5 min) followed by a linear gradient from 0 to 100 % solvent B in 30 min. Solvent B was then maintained at 100% for 5 min. The system was equilibrated for another 5 min at 100% of solvent A before the next sample injection. The mass spectra of various porphyrins and heme were obtained using MS (ThermoFinnigan's LCQ, SanJose, Calif.) coupled with electrospray ionization (ESI) interface. The spray voltage was 4.0 kV and the heated capillary temperature was 225° C. The amounts of individual porphyrins and heme were quantified using authentic porphyrins and heme purchased from Porphyrin Products Inc. (Logan, Utah) and ICN Biomedicals Inc. (Aurora, Ohio), respectively.

Effect of Metal Uptake Protein (ygiE) on Metalloporphyrins Production

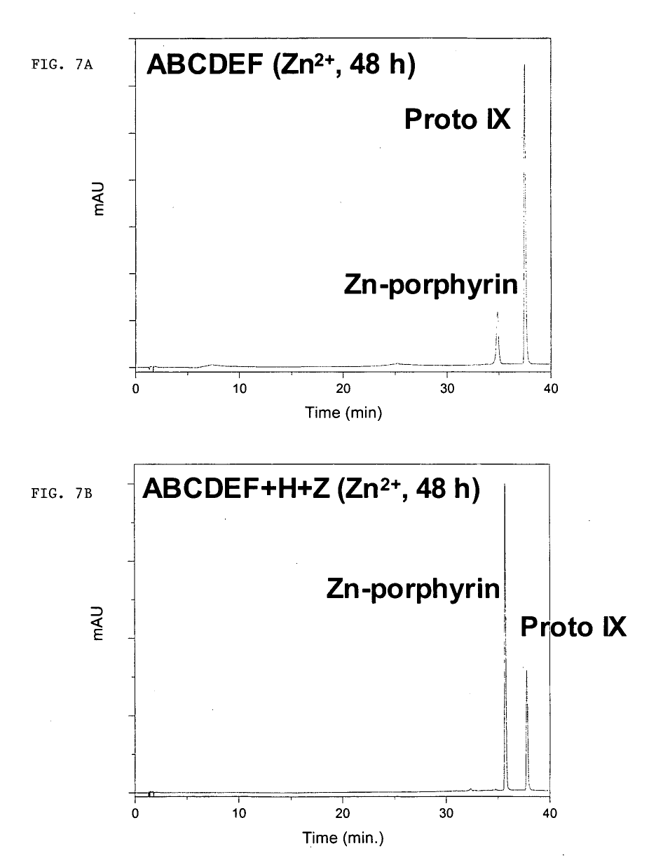

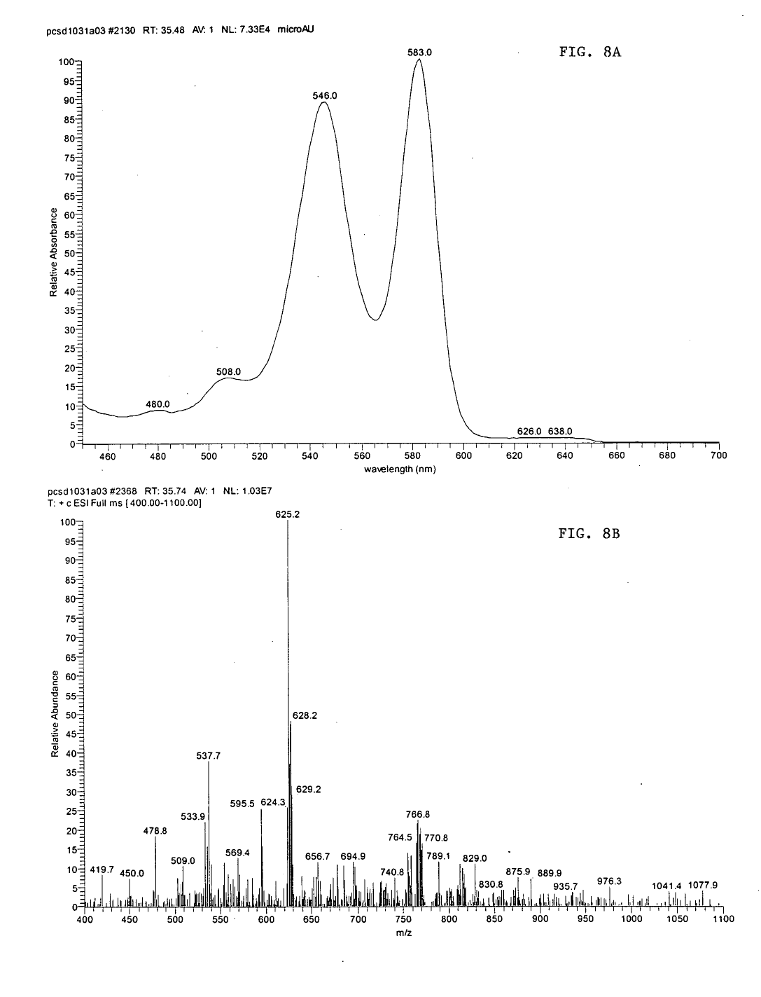

The zinc uptake system (ZupT) from E. coli shown to mediate zinc and other metal divalent ion uptake was cloned. In order to investigate the effect of ZupT for the production of metalloporphyrins in E. coli, zupT was coexpressed with hemABCDEFH. FIG. 7 and Table 5 show the HPLC analysis of E. coli transformants containing heme biosynthetic genes together with zupT and without zupT. FIG. 8 shows the Q band and ESI-Mass spectra of the zinc porphyrin peak separated by HPLC. The results show that the combined expression of hemH and zupT in E. coli significantly increases (more than three times) the production level of Zn-proto IX compared to E. coli transformants that do not overexpress the zupT gene. Interestingly, already the overexpression of ZupT in protoporphyrin accumulating transformants (hemABCDEF) that do not overexpress the ferrochelatase hemH increases Zn-protop IX production when compared to transformants expressing the complete heme-pathway including 15 the ferrochelatase but do not overexpress ZupT. These results show that ZupT was well expressed functionally, and zinc uptake is a limiting step for Zn-protop IX production in engineered E. coli.

TABLE 5
Effect of ZupT for the biosynthesis of Zn-protop IX in E. coli.

| | Area ratio × 100 |
|---|---|
| Genes expressed in E. coli | (Zn-protop IX/protoporphyrin IX) |
| hemABCDEF¹ + control²(24 h) | 5.6 |
| hemABCDEF + hemH(24 h) | 10.4 |
| hemABCDEF + zupT(24 h)³ | 11.8 |
| hemABCDEF + zupT + hemH(24 h) | 15.5 |
| hemABCDEF + control (Zn²⁺, 48 h) | 25.6 |
| hemABCDEF + hemH (Zn²⁺, 48 h) | 47.8 |
| hemABCDEF + zupT (Zn²⁺, 48 h) | 69.7 |
| hemABCDEF + zupT + hemH (Zn²⁺, | 165.9 |
| 48 h) | |

¹hemABCDEF means pACmod-hemABCD and pBBR1-hemEF.
²control means empty pUCmod vector only.
³hemABCDEF + zupT means pACmod-hemABCD, pBBR1-hemEFzupT, and pUCmod-hemH.

Area ratio was calculated from the corresponding peak areas obtained by HPLC analysis.

ZupT is a member of the ZIP (zinc and iron transporter) metal transporter family (Guerinot, 2000. Biophys.Acta 1465:190-198). ZupT was therefore also applied to produce heme in E. coli. Table 6 shows that ZupT indeed improved the production of heme in E. coli, which was similar to the production of Zn-protop IX. However, protoporphyrin IX still accumulates and is not completely converted to heme. This inefficient conversion of heme might be due to inefficient transport of ferrous ion by ZupT (i), low activity of iron-ferrochelatase (ii), and regulation of heme biosynthetic enzymes, uptake systems and/or biosynthetic precursor pathways by heme, as the final product of the pathway (iii).

TABLE 6
Effect of ZupT for the biosynthesis of heme in E. coli.

| | Area ratio × 100 (Heme/ |
|---|---|
| Genes expressed in E. coli | protoporphyrin IX) |
| hemABCDEF + control(24 h) | 5.7 |
| hemABCDEF + hemH(24 h) | 6.4 |
| hemABCDEF + zupT(24 h) | 10.7 |
| hemABCDEF + zupT + hemH(24 h) | 16.7 |
| hemABCDEF + control (Fe²⁺, 48 h) | 8.1 |
| hemABCDEF + hemH (Fe²⁺, 48 h) | 14.8 |
| hemABCDEF + zupT (Fe²⁺, 48 h) | 17.3 |
| hemABCDEF + zupT + hemH (Fe²⁺, 48 h) | 27.1 |

### Example 4

High-Throughput Screening of Ferrochelatase

To further increase metalloporphyrin production in engineered E. coli cells, directed evolution was used to obtain ferrochelatase variants for improved heme production. Directed evolution requires an efficient screen allowing to screen the thousands of mutants in a library. The laborious in vitro assay used for ferrochelatase activity measurement is not suitable for a high-throughput screen. To detect ferrochelatase activity in vivo, meaning in recombinant E. coli cells overexpressing the entire heme pathway, a time consuming an laborious HPLC analysis is necessary to detect the produced porphyrins and metalloporphyrins. To overcome these problems, a new high-throughput screening method based on FACS (fluorescent activated cell sorting) of E. coli cells overproducing porphyrins and metalloporphyrins was developed and used to screen a library of 10⁶ ferrochelatase variants.

Preparation of Mutant Ferrochelatase (hemH) Libraries.

A library of ferrochelatase mutants was created by error-prone PCR of the hemH gene from B. subtilis 168 (GenBank Accession No. Z99109). The final amplification products were ligated into pUCmod and transformed into protoporphyrin IX producing E. coli containing pACmod-hemABCD and pBBR-hemEFzupT.

FACS Analysis and Sorting of Mutant Ferrochelatase Libraries.

E. coli containing hem and zupT genes were cultivated for 24 hrs at 30° C. in 50 mL of LB. media containing carbenicillin (100 μg/ml), chloramphenicol (50 μg/ml), and kanamycin (30 μg/ml). After 24 hrs, 100 μM of ZnSO₄ and 1 mM of FeSO₄ were added into the media, respectively, and the bacterial cells were cultivated again at the same conditions for another 24 hrs. One hundred μL of the culture was suspended in 1 mL of PBS buffer, centrifuged, and the pellets were washed three times with PBS buffer. The cells washed were examined under a FACSCalibur (Becton Dickinson, Oxnard, Calif.). In order to screen mutant ferrochelatase libraries, the plasmid libraries (pUC-hemH library) were transformed into E. coli cells containing hemABCDEFH and zupT genes and cultured as described above. pUC-hemH168 (from B. subtilis) and pUC-hemH125 (from B. halodurans) were used as wild type controls. Sorting was performed in exclusion mode on about 10⁶ events at about 2000 events per second.

HPLC Analysis of the E. coli Cells Sorted.

The cells sorted by FACS were spread and cultivated on LB plate containing carbenicillin (100 μg/ml), chloramphenicol (50 μg/ml), and kanamycin (30 μg/ml) at 30° C. for 24 hrs. The colonies were randomly picked from the plate, cultivated in 50 mL of LB media containing the above three antibiotics and 1 mM FeSO₄ at 30° C. for 24 hrs. The cells were harvested by centrifugation (4,000×g, 20 minutes). The cell pellets were suspended with 50 mM EDTA solution in 100 mM of Tris-HCl buffer (pH 7.6) to remove the divalent ions added. Additionally, the cells were washed twice with Tris-HCl buffer (pH 7.6). The cells washed were extracted with acetone hydrochloric acid solution (acetone: 6 N HCl=10:1). The extracts were analyzed by the same method described as above.

FACS Analysis of E. coli Cells Producing (Metallo)Porphyrins.

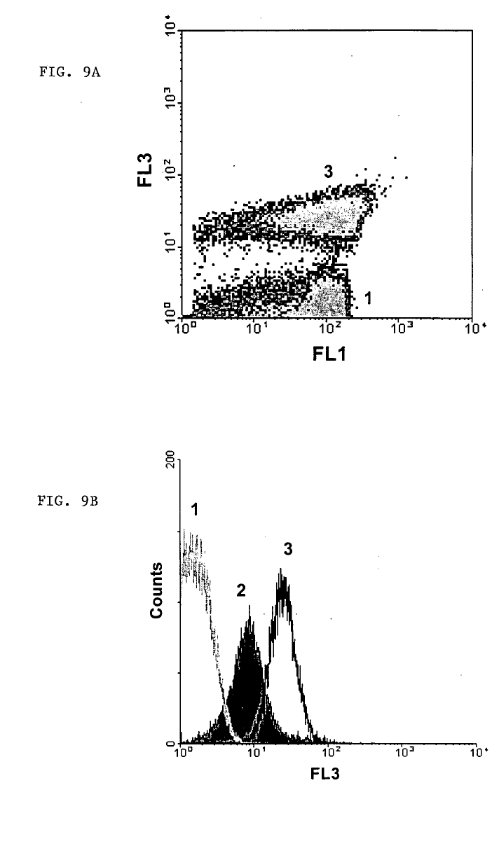

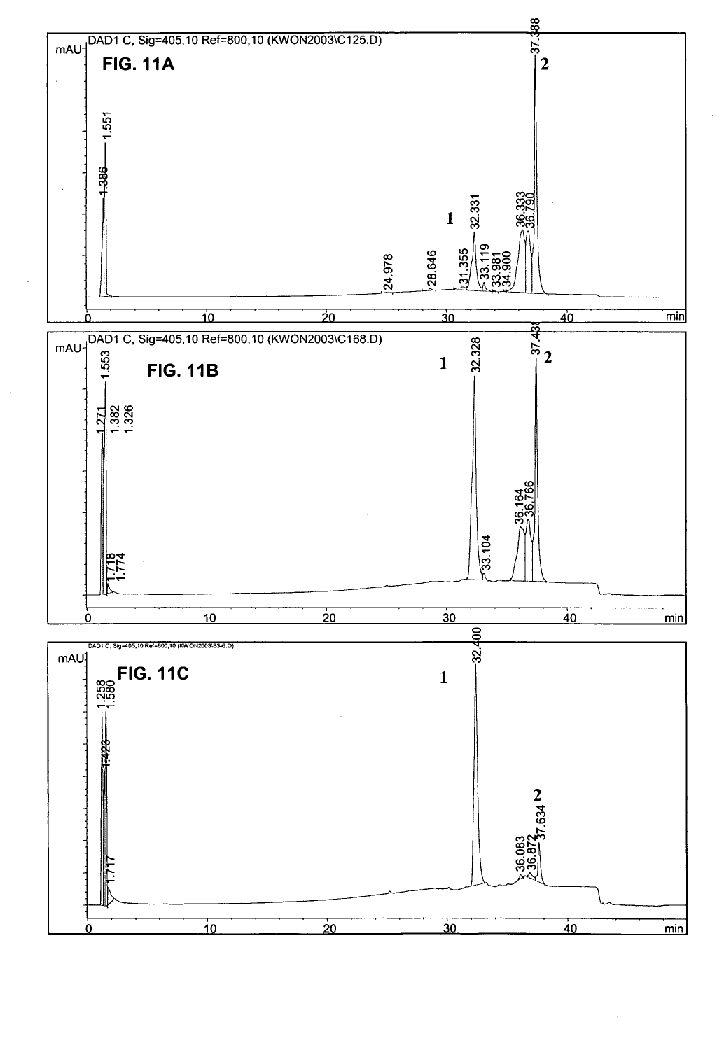

It was found that FACS analysis can discriminate between three different cell populations: (1) cells that are not producing porphyrins, (2) cells producing Zn-protoporphyrin IX, and (3) cells producing protoporphyrin IX (proto IX) in FL3 region (Em=650 nm) (FIG. 9). In particular, the population of proto IX producing E. coli cells was mostly red-shifted due to the fluorescence of proto IX (Eₘₐₓ=634 nm). However, the population of Zn-proto IX producing E. coli cells was relatively blue-shifted when compared to proto IX producing E. coli cells, which was the clear evidence of ferrochelatase activity (conversion of proto IX to Zn-proto IX).

Screening of Ferrochelatase (hemH) Library.

To find ferrochelatase mutants with better activity towards ferrous ion (Fe²⁺) when compared to the wild type, the gene encoding the ferrochelatase from B. subtilis 168 was subjected to error prone PCR and transformed into E. coli cells containing pACmod-hemABCD and pBBR-hemEFzupT. The transformants containing the hemH library were grown without ferrous ion at 30° C. for 24 hrs to produce proto IX sufficiently. After 24 hrs, iron (1 mM of Fe²⁺) was added into the media and the cells were grown for further 24 hrs. FIG. 10 shows FACS histograms of the E coli transformants containing wild type hemH168, hemH125, and the hemH library. The most blue-shifted region of the population 1 was sorted (FIG. 10). A total of 2.4×10⁶ cells were evaluated in 24 min, and 3052 individual clones were recovered. The cells sorted were isolated as single colonies on LB plates containing carbenicillin (l00 μg/ml), chloramphenicol (50 μg/ml), and kanamycin (30 μg/ml). Six clones were picked randomly, cultivated with 1 mM of FeSO₄ and analyzed by HPLC (Table 7, FIG. 1). These results show that the activity of the B. halodurans hemH was lower than the B. subtilis hemH, which correlated well with the FACS data (FIG. 10). Also, all mutants sorted showed higher heme production, which means higher ferrochelatase activity in vivo, when compared to wild type (Table 7). Therefore, the combination of fluorescence-based screening of proto IX producing E. coli cells is a very powerful method for high-throughput screening of ferrochelatases. DNA sequencing revealed that the six mutants screened by FACS had 1 to 7 base pair changes in the gene resulting in 1 to 3 amino acid substitutions (Table 8, numbering of residues based on hemH from B. subtilis 168). This method will be applicable to find new ferrochelatase mutants with new metal substrate specificities.

TABLE 7
Screening of iron-chelatase mutants by FACS sorting

| E. coli cells sorted | Area ratio × 100 (Heme/protoporphyrin IX) |
|---|---|
| HemH168 (wild type) | 131 |
| HemH125 (wild type) | 39.4 |
| Mutant 1 | 296 |
| Mutant 2 | 452 |
| Mutant 3 | 468 |
| Mutant 4 | 375 |
| Mutant 5 | 316 |
| Mutant 6 | 831 |

TABLE 8
Amino acid substitutions of six ferrochelatase mutants

| Mutant | Amino acid substitution(s) |
|---|---|
| Mutant 1 | T302A |
| Mutant 2 | D76G, K102T |
| Mutant 3 | E61K |
| Mutant 4 | M11V, G104A |
| Mutant 5 | R31G |
| Mutant 6 | E61K, L185Q, G212D |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A microorganism comprising at least one exogenous nucleic acid encoding a 5-aminolaevulinate (ALA) synthase polypeptide, an ALA dehydratase polypeptide, a porphobilinogen deaminase polypeptide, an uroporphyrinogen III synthase polypeptide, an uroporphyrinogen decarboxylase polypeptide, a coproporphyrinogen III oxidase polypeptide, a ferrochelatase polypeptide, and a metal ion transporter polypeptide, wherein said microorganism produces an amount of Zn-protoporphyrin IX or heme that is increased relative to that of a corresponding microorganism without the at least one exogenous nucleic acid.

2. The microorganism of claim 1, wherein said microorganism is a Gram negative or Gram positive bacteria.

3. The microorganism of claim 2, wherein said bacteria is Escherichia coli, a species of Pseudomonas, or a species of Propionibacterium.

4. The microorganism of claim 1, wherein the ferrous ion chelation activity of said ferrochelatase polypeptide is enhanced relative to a wild-type B. subtilis ferrochelatase polypeptide.

5. The microorganism of claim 4, wherein said ferrochelatase polypeptide comprises one or more mutations at amino acid residues selected from the group consisting of residues 11, 31, 61, 76, 102, 104, 184, 185, 212, and 302 of SEQ ID NO:7.

6. The microorganism of claim 5, wherein said ferrochelatase polypeptide comprises a mutation at residues 76 and 102 of SEQ ID NO:7.

7. The microorganism of claim 5, wherein said ferrochelatase polypeptide comprises a mutation at residues 11 and 104 of SEQ ID NO:7.

8. The microorganism of claim 5, wherein said ferrochelatase polypeptide comprises a mutation at residues 61, 185, and 212 of SEQ ID NO:7.

9. The microorganism of claim 1, wherein the metal ion transporter is a zinc and iron transporter.

10. The microorganism of claim 9, wherein said zinc and iron transporter is a ZupT polypeptide.


SEQUENCE LISTING (7 sequences)

SEQ ID NO: 1 - DNA, 62 bp, Artificial Sequence
  Primer
ggatgttaggatggaccacggatgataatgacggtaacaagcgtgtaggctggagctgcttc

SEQ ID NO: 2 - DNA, 62 bp, Artificial Sequence
  Primer
cctggtctcttcaaccacggcggaggttttttcttgttccgtcatatgaatatcctccttag

SEQ ID NO: 3 - DNA, 41 bp, Artificial Sequence
  Primer
gctctagaaggaggattacaaaatgtcagtacctctcattc

SEQ ID NO: 4 - DNA, 33 bp, Artificial Sequence
  Primer
taaagcggccgcttaaccaattcccgccgtttg

SEQ ID NO: 5 - DNA, 22 bp, Artificial Sequence
  synthetically generated insertion sequence
tgataaggaggtcgaccatatg

SEQ ID NO: 6 - DNA, 933 bp, Bacillus subtilis
atgagtagaaagaaaatggggcttctcgtgatggcgtacggcacgccttataaggaagaagatattgaacgttattatacacatatcagaagaggcagaaagcctgaacctgaaatgcttcaagatttgaaagacagatacgaagcgattggcggcatttcaccgcttgcccaaatcacagaacagcaggcgcataatctggaacagcacttgaatgaaattcaggatgagattacgtttaaagcgtatatcggactgaagcatatcgagccgtttattgaagacgccgtagcagagatgcataaggatggcattacagaagcagtcagcatcgtgctggcgccgcatttctccacgttcagcgttcagtcctacaacaaacgcgccaaagaagaggctgaaaaactcggcggcttaacgattacgtcagttgaaagctggtacgatgaaccgaagttcgtgacatattgggttgaccgagtgaaggaaacatatgcgtcaatgcctgaggatgaaagagaaaacgcaatgctcatcgtatccgcacacagcctgccggaaaaaatcaaagaatttggagacccgtacccggaccagcttcatgaatcggcgaaattaattgctgaaggcgcaggcgtttccgaatatgctgtcggctggcaaagtgaagggaacacgcctgatccttggctcggacccgatgttcaggatttaacacgtgatttattcgaacaaaaaggctatcaagcatttgtatatgttcctgttgggtttgtcgcggatcacttagaagtgctttatgataatgattatgaatgcaaagtggttactgacgatatcggcgcaagctattaccggccggaaatgccaaatgccaagcctgaatttattgatgctttggcaacagtcgtattaaaaaaattaggacgttaa

SEQ ID NO: 7 - protein, 310 aa, Bacillus subtilis
MSRKKMGLLVMAYGTPYKEEDIERYYTHIRRGRKPEPEMLQDLKDRYEAIGGISPLAQITEQQAHNLEQHLNEIQDEITFKAYIGLKHIEPFIEDAVAEMHKDGITEAVSIVLAPHFSTFSVQSYNKRAKEEAEKLGGLTITSVESWYDEPKFVTYWVDRVKETYASMPEDERENAMLIVSAHSLPEKIKEFGDPYPDQLHESAKLIAEGAGVSEYAVGWQSEGNTPDPWLGPDVQDLTRDLFEQKGYQAFVYVPVGFVADHLEVLYDNDYECKVVTDDIGASYYRPEMPNAKPEFIDALATVVLKKLGR